SEQUENCE LISTING (8 sequences)

SEQ ID NO: 1 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 2 - protein, 126 aa, Mus musculus
MKSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTT

SEQ ID NO: 3 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 4 - protein, 98 aa, Mus musculus
MNPSAAVIFCLILLGLSGTQGIPLARTVRCNCIHIDDGPVRMRAIGKLEIIPASLSCPRVEIIATMKKNDEQRCLNPESKTIKNLMKAFSQKRSKRAP

SEQ ID NO: 5 - DNA, 378 bp, Homo sapiens
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 6 - DNA, 381 bp, Mus musculus
atgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacataa

SEQ ID NO: 7 - DNA, 297 bp, Homo sapiens
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 8 - DNA, 297 bp, Mus musculus
atgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaa

(19) United States
(12) Patent Application Publication
(10) Pub. No.: US 2022/0280609 A1
(43) Pub. Date: Sep. 8, 2022

(54) COMBINATION CANCER THERAPY AGENTS AND METHODS

(71) Applicants: The Regents of the University of California, Oakland, CA (US); United States Government Represented by the Department of Veterans Affairs, Washington, DC (US)

(72) Inventors: Steven M. DUBINETT, Los Angeles, CA (US); Bin LIU, Woodland Hills, CA (US); Raymond LIM, Tustin, CA (US); Ramin SALEHI-RAD, Encino, CA (US)

(73) Assignees: The Regents of the University of California, Oaklandca (US); United States Government Represented by the Department of Veterans Affairs, Washington, DC (US)

(21) Appl. No.: 17/625,062

(22) PCT Filed: Jul. 17, 2020
(86) PCT No.: PCT/US20/42453

§ 371 Date: Jan. 5, 2022

Related U.S. Application Data
(60) Provisional application No. 62/875,230, filed on Jul. 17, 2019.

(51) Int. Cl. A61K 38/19; C07K 16/28; A61P 35/00; A61K 45/06

(52) U.S. Cl. CPC A61K 38/195; C07K 16/2818; A61P 35/00; A61K 45/06

(57) ABSTRACT

The present disclosure relates, in general, to methods for treating cancer comprising administering to a subject in need thereof an effective amount of CXCL9, CXCL10 or the combination, in combination with an immune checkpoint inhibitor. The CXCL9, CXCL10 or combination may be administered as a polypeptide, a polynucleotide or cells comprising a polynucleotide encoding CXCL9, CXCL10 or both. In one aspect, the treatment is amenable to patients with low or high mutational burden tumors.

### GOVERNMENT INTEREST STATEMENT

This work was supported by the U.S. Department of Veterans Affairs, and the Federal Government has certain rights in the invention.

### BACKGROUND OF THE INVENTION

Lung cancer is the most common cause of cancer death worldwide with approximately 85% of patients having non-small cell lung cancer (NSCLC). Recent approval of chemo-immunotherapy combinations has altered the treatment landscape in advanced NSCLC, such that all eligible patients can receive anti-PD-1/PD-L1 immunotherapy in the front-line setting, either in combination with chemotherapy irrespective of tumor PD-L1 level, or monotherapy in select patients with tumor PD-L1>50%. This shift has led to improved patient outcomes in the front-line setting [the objective response rate (ORR) is approximately 55%], but also created an area of unmet need, namely effective treatment options for patients after progression on a PD-1 and/or PD-L1 inhibitor. Second-line chemotherapy is an option for these patients but is associated with significant toxicity and limited efficacy. As an alternative, treatment with a PD-1 or PD-L1 inhibitor beyond progression has been evaluated with minimal clinical benefit observed (the ORR is approximately 8%).

Studies reveal that responses to PD-1/PD-L1 blockade are associated with high tumor mutational burden (TMB), increased CD8+ T cell tumor infiltration and high baseline tumor PD-L1 expression. In contrast, oncogenic driver mutations may contribute to resistance to anti-PD-1/PD-L1 immunotherapy. For example, LKB1 inactivating mutations have been implicated in driving primary resistance to anti-PD-1 therapy in KRAS-mutant lung adenocarcinoma (LUAC), probably by facilitating an immunosuppressive and angiogenic environment to promote tumor growth. In addition, patients with epidermal growth factor receptor (EGFR) mutations and/or anaplastic lymphoma kinase (ALK) rearrangements do not benefit from PD-1/PD-L1 inhibition after failed tyrosine kinase inhibitor (TKI) therapy. EGFR and/or ALK-mutant tumors often harbor uninflamed and immunosuppressive TME, which may contribute to their resistance to immunotherapy. Therefore, approaches that enhance tumor antigen presentation, overcome the immunosuppressive TME and inhibit tumor angiogenesis are anticipated to lead to improved efficacy of PD-1/PD-L1 blockade.

It is towards improving the efficacy of PD-1/PD-L1 blockade, and resistance to immune checkpoint inhibitor therapy in general, that the present invention is directed.

### SUMMARY OF THE INVENTION

In one aspect, a method of treating cancer or a solid tumor in a subject is provided comprising (a) administering to the subject (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) any combination thereof; and (b) administering to the subject an immune checkpoint inhibitor.

In one embodiment, the immune checkpoint inhibitor is selected from the group consisting of a CTLA-4 inhibitor, a CTLA-4 receptor inhibitor, a PD-1 inhibitor, a PD-L1 inhibitor, a Pall inhibitor, a PD1-L2 inhibitor, a 4-1BB inhibitor, an OX40 inhibitor, a LAG-3 inhibitor, a TIM-3 inhibitor, or a combination thereof. In one embodiment, the immune checkpoint inhibitor is an antibody, optionally, a monoclonal antibody. In one embodiment, the immune checkpoint inhibitor is a CTLA-4 inhibitor, optionally, ipilimumab or tremilimumab.

In one embodiment, the immune checkpoint inhibitor is a PD1 inhibitor selected from a group consisting of: Nivolumab, Pembrolizumab, Pidilizumab, Lambrolizumab, BMS-936559, Atezolizumab, and AMP-224, AMP224, AUNP12, BGB108, MCLA134, MEDI0680, PDR001, REGN2810, SHR1210, STIAI10X, STIA1110 and TSR042.

In one embodiment, the immune checkpoint inhibitor is a PD1-L1 inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

In one embodiment, CXCL9/10 refers to and comprises CXCL9, CXCL10, or the combination thereof. In one embodiment of the combination, each of CXCL9 and CXCL10 is administered in a different form, each independently as a polypeptide, a polynucleotide, as a cell comprising the polynucleotide, or any combination thereof. In one embodiment the CXCL9 is administered as a polypeptide. In one embodiment CXCL10 is administered as a polypeptide. In one embodiment, the combination of CXCL9 and CXCL10 are administered, each as polypeptides. In one embodiment the CXCL9 is administered as a polynucleotide encoding CXCL9. In one embodiment CXCL10 is administered as a polynucleotide encoding CXCL10. In one embodiment, the combination of CXCL9 and CXCL10 are administered, each as polynucleotides encoding CXCL9 and CXCL10, respectively. In one embodiment the CXCL9 is administered as a cell comprising the polynucleotide encoding CXCL9. In one embodiment the CXCL10 is administered as a cell comprising the polynucleotide encoding CXCL10. In one embodiment the combination of CXCL9 and CXCL10 are administered, as a cell comprising the polynucleotide encoding CXCL9, and a cell encoding the polynucleotide encoding CXCL10.

In one embodiment, the combination of CXCL9 and CXCL10 are administered, CXCL9 is administered as a polypeptide and CXCL10 is administered as a polynucleotide encoding CXCL10. In one embodiment, the combination of CXCL9 and CXCL10 are administered, CXCL10 is administered as a polypeptide and CXCL9 is administered as a polynucleotide encoding CXCL9. In one embodiment, the combination of CXCL9 and CXCL10 are administered, CXCL9 is administered as a polypeptide and CXCL10 is administered as a cell comprising a polynucleotide encoding CXCL10. In one embodiment, the combination of CXCL9 and CXCL10 are administered, CXCL10 is administered as a polypeptide and CXCL9 is administered as a cell comprising a polynucleotide encoding CXCL9. In one embodiment, the combination of CXCL9 and CXCL10 are administered, CXCL9 is administered as a polynucleotide encoding CXCL9, and CXCL10 is a administered as a cell comprising a polynucleotide encoding CXCL10. In one embodiment, the combination of CXCL9 and CXCL10 are administered, CXCL10 is administered as a polynucleotide encoding CXCL10, and CXCL9 is a administered as a cell comprising a polynucleotide encoding CXCL9.

In one embodiment, the CXCL9 polypeptide comprises an amino acid sequence of SEQ ID NO: 1 or SEQ ID NO: 2. In one embodiment, the CXCL9 polypeptide consists of an amino acid sequence of SEQ ID NO: 1 or SEQ ID NO: 2. In one embodiment, the polynucleotide encoding CXCL9 polypeptide comprises the sequence of SEQ ID NO: 5 or SEQ ID NO: 6. In one embodiment, the polynucleotide encoding CXCL9 polypeptide consists of the sequence of SEQ ID NO: 5 or SEQ ID NO: 6. In one embodiment, the CXCL10 polypeptide comprises an amino acid sequence of SEQ ID NO: 3 or SEQ ID NO: 4. In one embodiment, the CXCL10 polypeptide consists of an amino acid sequence of SEQ ID NO: 3 or SEQ ID NO: 4. In one embodiment, the polynucleotide encoding CXCL10 polypeptide comprises the sequence of SEQ ID NO: 7 or SEQ ID NO: 8. In one embodiment, the polynucleotide encoding CXCL10 polypeptide consists of the sequence of SEQ ID NO: 7 or SEQ ID NO: 8. In one embodiment, a cell comprising the polynucleotide encoding the CXCL9 polypeptide comprises the sequence of SEQ ID NO:5 or SEQ ID NO:6. In one embodiment, a cell comprising the polynucleotide encoding the CXCL10 polypeptide comprises the sequence of SEQ ID NO:7 or SEQ ID NO:8. In one embodiment, a cell comprising the polynucleotide encoding the CXCL9 polypeptide and the CXCL10 polypeptide comprises the sequence of SEQ ID NO:5 or SEQ ID NO:6, and SEQ ID NO:7 or SEQ ID NO:8.

In one embodiment, CXCL9/10 in any form described herein, and the immune checkpoint inhibitor are independently administered by a route selected from intratumorally, intravenously, intra-arterially, intraperitoneally, intranasally, intramuscularly, intradermally or subcutaneously, or via CT-guided or bronchoscopic IT injection. In one embodiment, the CXCL9/10 is administered intratumorally and the immune checkpoint inhibitor is administered intravenously.

In one embodiment, the polynucleotide encoding the CXCL9 polypeptide, CXCL10 polypeptide, or the combination thereof is inserted into a vector and the vector is administered to the subject, or the vector is introduced into an antigen presenting cell (APC) or a dendritic cell (DC) which is then administered to the subject or to the tumor site. In one embodiment, the vector is an adenovirus vector, a lentiviral vector, a CMV vector, a vaccinia virus vector, a sindbis virus vector, or a herpesvirus vector. In one embodiment, the adenoviral vector is a replication-deficient adenoviral vector. In one embodiment, the cell comprising the polynucleotide encoding the CXCL9 polypeptide, CXCL10 polypeptide, or the combination thereof is an antigen presenting cell (APC) or a dendritic cell (DC). In one embodiment, the APC is a dendritic cell. In one embodiment, the dendritic cell is autologous to the subject. In one embodiment the dendritic cell is from a donor. In one embodiment the dendritic cell is from a cell line.

In one embodiment, at least or about 1×10\hat{ }6 cells comprising the polynucleotide encoding the CXCL9 polypeptide, CXCL10 polypeptide, or about 1×10\hat{ }6 cells each in the case of the combination thereof, are administered to the subject. In one embodiment, the cells produce at least or about 10 ng of each of CXCL9 and CXCL10 per 1×10\hat{ }6 cells in a 24-hour period. In one embodiment, at least or about 1×10\hat{ }6 cells comprising the polynucleotide encoding the CXCL9 polypeptide and about 1×10\hat{ }6 cells comprising the polynucleotide encoding the CXCL10 polypeptide are administered to the subject. In one embodiment, the CXCL9 expressing cells produce at least or about 10 ng of CXCL9 per 1×10\hat{ }6 cells in a 24-hour period, and the CXCL10 expressing cells produce at least or about 10 ng of CXCL10 per 1×10\hat{ }6 cells in a 24-hour period.

In one embodiment, the subject comprises a solid tumor and the polypeptides, polynucleotides or cells, or any combination thereof, are administered to the subject intratumorally. In one embodiment, the solid tumor is a non-small cell lung carcinoma (NSCLC) solid tumor.

In one embodiment, the (i) CXCL9 polypeptide, CXCL10 polypeptide, or the combination thereof, (ii) polynucleotide encoding the CXCL9 polypeptide, CXCL10 polypeptide, or the combination thereof, (iii) cell comprising the polynucleotide encoding the CXCL9 polypeptide, CXCL10 polypeptide, or the combination thereof, or (iv) any combination thereof, is administered to the subject prior to, about 2 weeks prior to, or at the same time as the immune checkpoint inhibitor. In one embodiment, the (i) CXCL9 polypeptide, CXCL10 polypeptide, or the combination thereof, (ii) polynucleotide encoding the CXCL9 polypeptide, CXCL10 polypeptide, or the combination thereof, (iii) cell comprising the polynucleotide encoding the CXCL9 polypeptide, CXCL10 polypeptide, or the combination thereof, or (iv) combination thereof, is administered to the subject about more than once, once every two weeks, once every three weeks, or once a month. In one embodiment, the immune checkpoint inhibitor is administered to the subject more than once, once every 2 weeks, once every 3 weeks, or once a month. In some embodiments, the administration of each of the CXCL9/10 and the immune checkpoint inhibitor comprises multiple administrations of each during a 2, 3 or 4 week period, a rest period, then a repeat of the same regimen. Two or more such cycles may be administered, in other embodiments.

In one embodiment, the (i) CXCL9 polypeptide, CXCL10 polypeptide, or the combination thereof, (ii) polynucleotide encoding the CXCL9 polypeptide, CXCL10 polypeptide, or the combination thereof, (iii) cell comprising the polynucleotide encoding the CXCL9 polypeptide, CXCL10 polypeptide, or the combination thereof, or (iv) combination thereof, is administered intratumorally to the subject on days 7, 11 and 15, and the checkpoint inhibitor on days 7, 9, 11, 13 and 15. In one embodiment, the (i) CXCL9 polypeptide, CXCL10 polypeptide, or the combination thereof, (ii) polynucleotide encoding the CXCL9 polypeptide, CXCL10 polypeptide, or the combination thereof, (iii) cell comprising the polynucleotide encoding the CXCL9 polypeptide, CXCL10 polypeptide, or the combination thereof, or (iv) combination thereof, is administered intratumorally to the subject on days 7, 10 and 13, and the checkpoint inhibitor on days 7, 10, 13 and 15. In one embodiment, the dosing regiment is repeated one or in multiple cycles, with a rest between cycles. In one embodiment the regimen is repeated every 2 weeks. In one embodiment the cycle is repeated every 3 weeks. In one embodiment the cycle is repeated every month.

In one embodiment, a method is provided for treating cancer or a solid tumor having a high mutational burden in a subject comprising a. administering to the subject: (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) any combination thereof; and b. administering to the subject an immune checkpoint inhibitor.

In one embodiment, a method is provided for treating cancer or reducing the reoccurrence of a high mutational burden cancer in a subject in need thereof comprising administering an effective amount of a combination therapy comprising a) dendritic cells comprising an CXCL9/10 vector construct on days 0, 21, and 42, and b) an effective amount of anti-PD-1 antibody every three weeks starting on day 0, optionally wherein the vector is an lentiviral vector.

In one embodiment, a method is provided for treating cancer or a solid tumor having a low mutational burden in a subject comprising a. administering to the subject: (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) any combination thereof; and b. administering to the subject an immune checkpoint inhibitor.

In one embodiment, a method is provided for treating cancer or reducing the reoccurrence of a low mutational burden cancer in a subject in need thereof comprising administering an effective amount of a combination therapy comprising a) dendritic cells comprising an CXCL9/10 vector construct on days 0, 21, and 42, and b) an effective amount of anti-PD-1 antibody every three weeks starting on day 0, optionally wherein the vector is an lentiviral vector.

In one embodiment, a kit is provided comprising (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) a combination thereof, and an immune checkpoint inhibitor. In one embodiment, the immune checkpoint inhibitor is selected from the group consisting of a CTLA-4 inhibitor, a CTLA-4 receptor inhibitor, a PD-1 inhibitor, a PD-L1 inhibitor, a PD1-L2 inhibitor, a 4-1BB inhibitor, an OX40 inhibitor, a LAG-3 inhibitor, a TIM-3 inhibitor, or a combination thereof. In one embodiment, the immune checkpoint inhibitor is an antibody, optionally, a monoclonal antibody. In one embodiment, the immune checkpoint inhibitor is a CTLA-4 inhibitor, optionally, ipilimumab or tremilimumab. In one embodiment, the immune checkpoint inhibitor is a PD1 inhibitor selected from a group consisting of: nivolumab, pembrolizumab, pidilizumab, lambrolizumab, BMS-936559, atezolizumab, AMP-224, AMP224, AUNP12, BGB108, MCLA134, MEDI0680, PDR001, REGN2810, SHR1210, STIA110X, STIA1110 and TSR042. In one embodiment, the immune checkpoint inhibitor is a PD1-LI inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

In one embodiment of the kit, the CXCL9/10 comprises CXCL9, CXCL10, or the combination thereof. In one embodiment, the CXCL9 polypeptide comprises an amino acid sequence of SEQ ID NO: 1 or SEQ ID NO: 2; and the CXCL10 polypeptide comprises an amino acid sequence of SEQ ID NO: 3 or SEQ ID NO: 4. In one embodiment, the CXCL9 polypeptide consists of an amino acid sequence of SEQ ID NO: 1 or SEQ ID NO: 2; and the CXCL10 polypeptide consists of an amino acid sequence of SEQ ID NO: 3 or SEQ ID NO: 4. In one embodiment, the polynucleotide encoding CXCL9 polypeptide comprises the sequence of SEQ ID NO: 5 or SEQ ID NO: 6. In one embodiment, the polynucleotide encoding CXCL9 polypeptide consists of the sequence of SEQ ID NO: 5 or SEQ ID NO: 6. In one embodiment, the polynucleotide encoding CXCL10 polypeptide comprises the sequence of SEQ ID NO: 7 or SEQ ID NO: 8. In one embodiment, the polynucleotide encoding CXCL10 polypeptide consists of the sequence of SEQ ID NO: 7 or SEQ ID NO: 8. In one embodiment, a cell comprising the polynucleotide encoding the CXCL9 polypeptide comprises the sequence of SEQ ID NO:5 or SEQ ID NO:6. In one embodiment. a cell comprising the polynucleotide encoding the CXCL10 polypeptide comprises the sequence of SEQ ID NO:7 or SEQ ID NO:8. In one embodiment, a cell comprising the polynucleotide encoding the CXCL9 polypeptide and the CXCL10 polypeptide comprises the sequence of SEQ ID NO:5 or SEQ ID NO:6, and SEQ ID NO:7 or SEQ ID NO:8.

In one embodiment, a dendritic cell is provided comprising a vector that comprises a polynucleotide encoding CXCL9/10. In one embodiment, the CXCL9/10 comprises CXCL9, CXCL10, or the combination thereof. In one embodiment, the CXCL9 polypeptide comprises an amino acid sequence of SEQ ID NO: 1 or SEQ ID NO: 2; and the CXCL10 polypeptide comprises an amino acid sequence of SEQ ID NO: 3 or SEQ ID NO: 4. In one embodiment, the CXCL9 polypeptide consists of an amino acid sequence of SEQ ID NO: 1 or SEQ ID NO: 2; and the CXCL10 polypeptide consists of an amino acid sequence of SEQ ID NO: 3 or SEQ ID NO: 4. In one embodiment, the polynucleotide encoding CXCL9 polypeptide comprises the sequence of SEQ ID NO: 5 or SEQ ID NO: 6. In one embodiment, the polynucleotide encoding CXCL9 polypeptide consists of the sequence of SEQ ID NO: 5 or SEQ ID NO: 6. In one embodiment, the polynucleotide encoding CXCL10 polypeptide comprises the sequence of SEQ ID NO: 7 or SEQ ID NO: 8. In one embodiment, the polynucleotide encoding CXCL10 polypeptide consists of the sequence of SEQ ID NO: 7 or SEQ ID NO: 8. in one embodiment, a cell comprising the polynucleotide encoding the CXCL9 polypeptide comprises the sequence of SEQ ID NO:5 or SEQ ID NO:6. In one embodiment, a cell comprising the polynucleotide encoding the CXCL10 polypeptide comprises the sequence of SEQ ID NO:7 or SEQ ID NO:8. In one embodiment, a cell comprising the polynucleotide encoding the CXCL9 polypeptide and the CXCL10 polypeptide comprises the sequence of SEQ ID NO:5 or SEQ ID NO:6, and SEQ ID NO:7 or SEQ ID NO:8.

### BRIEF DESCRIPTION OF THE DRAWINGS

FIG. 1A, FIG. 1B and FIG. 1C show that genetically engineered murine models (GEMMs) of lung cancer bearing varying mutational loads recapitulate the clinical response.

FIG. 2A, FIG. 2B and FIG. 2C show the distinct tumor microenvironment (TME) immune phenotypes of the three genetic subtypes of GEMMs.

FIG. 3A and FIG. 3B show that the combination of CXCL9/10-DCs and anti-PD-1 outperforms monotherapies.

FIG. 4A and FIG. 4B show the correlation between CXCL9/10 gene expression and CD8+ T cells and dendritic cells in The Cancer Genome Atlas (TCGA) data.

FIG. 5A, FIG. 5B and FIG. 5C show the experimental design and results of a study showing that that the combination of intratumoral CXCL9/10-DCs and anti-PD-1 outperforms monotherapies.

FIG. 6 shows that intratumoral CXCL9/10-DC potentiates anti-PD-1 efficacy in the KPL-3M model.

FIG. 7A, FIG. 7B and FIG. 7C show that CXCL9-DC and CXCL10-DC are functionally equivalent in potentiating the antitumor efficacy of anti-PD-1, based on tumor volume.

FIG. 8A, FIG. 8B and FIG. 8C show that CXCL9-DC and CXCL10-DC are functionally equivalent in potentiating the antitumor efficacy of anti-PD-1, based on tumor weight.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. For the uses in non-humans, the species appropriate sequences (polypeptide, polynucleotide) of CXCL9 and/or CXCL10 are used; for antibodies to immune checkpoint inhibitors, antibodies to the species appropriate immune checkpoint. For cell based delivery, the species appropriate cells are used. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

One approach to overcome the immunosuppressive tumor microenvironment (TME) is to utilize in situ vaccination with chemokine gene-modified functional antigen presenting cells (APCs) to enhance tumor antigen presentation and promote tumor-specific T cell activation. It has been shown that the chemokines CXCL9 and CXCL10 are important signaling molecules secreted by CD103+ dendritic cells (DCs) to recruit effector T cells and orchestrate effective anti-tumor immunity. Furthermore, CXCL9/10 (i.e., one or the combination of CXCL9 and CXCL10) have anti-angiogenic properties, which can shift the angiogenic balance from tumor-induced angiogenesis to angiostasis. Thus, in one embodiment, a method of treatment is described herein using IT injection of CXCL9/10-secreting DCs (CXCL9/10-DCs) to potentiate the response to anti-PD-1 therapy. In one embodiment an immune checkpoint inhibitor such as but not limited to anti-PD-1 therapy is co-administered. Using a genetically engineered murine model (GEMM) of lung cancer bearing increased tumor mutational burden (TMB) recapitulating the mutational landscape of clinical NSCLC, the efficacy of the cell-based therapy and the combination therapy are demonstrated. In situ vaccination with CXCL9/10-DC may become an effective approach to sensitize non-responsive NSCLC to anti-PD-1/PD-L1 immunotherapy. Efficacy in low mutational burden tumors as well as in high mutational burden tumors is provided.

### CXCL9/10

CXCL9 and CXCL10 useful in the methods of the invention encompass both naturally occurring polypeptides as well as variations and modified forms thereof.

CXCL9/10 as used herein refers to the chemokines CXCL9 or CXCL10, or their combination, and may refer to them individually or combination in the form of polypeptides, polynucleotides, or cells comprising one or both polynucleotides and/or expressing one or both polypeptides. CXCL9/10 also refers to any combination of any of the foregoing, such as the combination of CXCL9 and CXCL10 polypeptides, the combination of CXCL9 and CXCL10 polynucleotides, the combination of cells, one comprising the CXCL9 polynucleotide and one comprising the CXCL10 polynucleotides; and in other combinations, such as CXCL9 polypeptide and a cell expressing CXCL10, CXCL10 polypeptide and a cell comprising CXCL9 polynucleotide. Moreover, each of CXCL9 or CXCL10 in any of the polypeptide, polynucleotide or cell forms may be used individually in the practice of the invention, or in combination.

Chemokine (C-X-C motif) ligand 9 (CXCL9) is a small cytokine (11.7 kDa) belonging to the CXC chemokine family that is also known as Monokine induced by gamma interferon (MIG). CXCL9 is one of the chemokines that plays role to induce chemotaxis, promote differentiation and multiplication of leukocytes, and cause tissue extravasation. The human CXCL9 chemokine amino acid sequence is depicted in SEQ ID NO:1. The murine CXCL9 chemokine amino acid sequence is depicted in SEQ ID NO:2.

The amino acid sequence for human CXCL9 is:

(SEQ ID NO: 1)

MKKSGVLFLL GIILLVLIGV QGTPVVRKGR CSCISTNQGT
IHLQSLKDLK QFAPSPSCEK IEIIATLKNG VQTCLNPDSA
DVKELIKKWE KQVSQKKKQK NGKKHQKKKV LKVRKSQRSR
QKKTT.

The amino acid sequence for murine CXCL9 is:

(SEQ ID NO: 2)

MKSAVLFLLG IIFLEQCGVR GTLVIRNARC SCISTSRGTI
HYKSLKDLKQ FAPSPNCNKT EIIATLKNGD QTCLDPDSAN
VKKLMKEWEK KISQKKKQKR GKKHQKNMKN RKPKTPQSRR
RSRKTT.

In one non-limiting example, a polynucleotide encoding human CXCL9 has the sequence:

(SEQ ID NO: 5)
ATGAAGAAAAGTGGTGTTCTTTTCCTCTTGGGCATCATCTTGCTGGTTCT
GATTGGAGTGCAAGGAACCCCAGTAGTGAGAAAGGGTCGCTGTTCCTGCA
TCAGCACCAACCAAGGGACTATCCACCTACAATCCTTGAAAGACCTTAAA
CAATTTGCCCCAAGCCCTTCCTGCGAGAAAATTGAAATCATTGCTACACT
GAAGAATGGAGTTCAAACATGTCTAAACCCAGATTCAGCAGATGTGAAGG
AACTGATTAAAAAGTGGGAGAAACAGGTCAGCCAAAAGAAAAAGCAAAAG
AATGGGAAAAAACATCAAAAAAAGAAAGTTCTGAAAGTTCGAAAATCTCA
ACGTTCTCGTCAAAAGAAGACTACATAA.

In one non-limiting example, a polynucleotide encoding murine CXCL9 has the sequence:

(SEQ ID NO: 6)
ATGAAGTCCGCTGTTCTTTTCCTCTTGGGCATCATCTTCCTGGAGCAGTG
TGGAGTTCGAGGAACCCTAGTGATAAGGAATGCACGATGCTCCTGCATCA
GCACCAGCCGAGGCACGATCCACTACAAATCCCTCAAAGACCTCAAACAG
TTTGCCCCAAGCCCCAATTGCAACAAAACTGAAATCATTGCTACACTGAA
GAACGGAGATCAAACCTGCCTAGATCCGGACTCGGCAAATGTGAAGAAGC
TGATGAAAGAATGGGAAAAGAAGATCAGCCAAAAGAAAAAGCAAAAGAGG
GGGAAAAAACATCAAAAGAACATGAAAAACAGAAAACCCAAAACACCCCA
AAGTCGTCGTCGTTCAAGGAAGACTACATAA.

C-X-C motif chemokine 10 (CXCL10) also known as interferon gamma-induced protein 10 (IP-10) or small-inducible cytokine B 10, is an 8.7 kDa protein that in humans is encoded by the CXCL10 gene. The human CXCL10 chemokine amino acid sequence is depicted in SEQ ID NO:3. The murine CXCL10 chemokine amino acid sequence is depicted in SEQ ID NO:4.

The amino acid sequence of human CXCL10 is:

(SEQ ID NO: 3)

MNQTAILICC LIFLTLSGIQ GVPLSRTVRC TCISISNQPV
NPRSLEKLEI IPASQFCPRV EIIATMKKKG EKRCLNPESK
AIKNLLKAVS KERSKRSP

The amino acid sequence of murine CXCL10 is:

(SEQ ID NO: 4)
MNPSAAVIFC LILLGLSGTQ GIPLARTVRC NCIHIDDGPV
RMRAIGKLEI IPASLSCPRV EIIATMKKND EQRCLNPESK
TIKNLMKAFS QKRSKRAP

In one non-limiting example, a polynucleotide encoding human CXCL10 has the sequence:

(SEQ ID NO: 7)
ATGAATCAAACTGCCATTCTGATTTGCTGCCTTATCTTTCTGACTCTAAG
TGGCATTCAAGGAGTACCTCTCTCTAGAACTGTACGCTGTACCTGCATCA
GCATTAGTAATCAACCTGTTAATCCAAGGTCTTTAGAAAAACTTGAAATT
ATTCCTGCAAGCCAATTTTGTCCACGTGTTGAGATCATTGCTACAATGAA
AAAGAAGGGTGAGAAGAGATGTCTGAATCCAGAATCGAAGGCCATCAAGA
ATTTACTGAAAGCAGTTAGCAAGGAAAGGTCTAAAAGATCTCCTTAA.

In one non-limiting example, a polynucleotide encoding murine CXCL10 has the sequence:

(SEQ ID NO: 8)
ATGAACCCAAGTGCTGCCGTCATTTTCTGCCTCATCCTGCTGGGTCTGAG
TGGGACTCAAGGGATCCCTCTCGCAAGGACGGTCCGCTGCAACTGCATCC
ATATCGATGACGGGCCAGTGAGAATGAGGGCCATAGGGAAGCTTGAAATC
ATCCCTGCGAGCCTATCCTGCCCACGTGTTGAGATCATTGCCACGATGAA
AAAGAATGATGAGCAGAGATGTCTGAATCCGGAATCTAAGACCATCAAGA
ATTTAATGAAAGCGTTTAGCCAAAAAAGGTCTAAAAGGGCTCCTTAA

CXCL9 and CXCL10 include naturally occurring mammalian CXCL9 and CXCL10, and variants and fragments thereof. Preferably the CXCL9 and CXCL10 are of human or mouse origin. Most preferably the CXCL9 and CXCL10 are human CXCL9 and CXCL10.

CXCL9 and CXCL10 polypeptides for use in the methods disclosed herein can be CXCL9 or CXCL10 variants, CXCL9 or CXCL10 fragments, analogues, and derivatives.

As described elsewhere, each of these chemokines, in the form of a polypeptide, a polynucleotide, a cell comprising a polynucleotide, of either CXCL9 or CXCL10 or both, may be administered to the subject together with the checkpoint inhibitor. For example, CXCL9 may be the only chemokine used in the methods described herein, and may be administered as a polypeptide, polynucleotide or cell comprising the polynucleotide, or any combination thereof. In another example, CXCL10 may be the only chemokine used in the methods described herein, and may be administered as a polypeptide, polynucleotide or cell comprising the polynucleotide, or any combination thereof. In another example, both CXCL9 and CXCL10 are used in the methods described here but they may be administered independently as a polypeptide, polynucleotide or cell comprising a polynucleotide, or any combination thereof. In one example, CXCL9 is administered intratumorally as a polypeptide, and CXCL10 administered intratumorally as a cell comprising a polynucleotide encoding CXCL10. In one embodiment, two or three means of delivery may be used for one or both chemokines, for example, the polypeptide and the polynucleotide, or the polypeptide and the cell, or the polynucleotide and the cell. In other embodiments, any combination of means of delivery, routes of delivery, and dosing schedules of either or both chemokines (in any form) is embraced herein.

In one embodiment, the CXCL9 polypeptide comprises an amino acid sequence of SEQ ID NO: 1 or SEQ ID NO: 2. In one embodiment, the CXCL9 polypeptide consists of an amino acid sequence of SEQ ID NO: 1 or SEQ ID NO: 2. In one embodiment, the polynucleotide encoding CXCL9 polypeptide comprises the sequence of SEQ ID NO: 5 or SEQ ID NO: 6. In one embodiment, the polynucleotide encoding CXCL9 polypeptide consists of the sequence of SEQ ID NO: 5 or SEQ ID NO: 6. In one embodiment, the CXCL10 polypeptide comprises an amino acid sequence of SEQ ID NO: 3 or SEQ ID NO: 4. In one embodiment, the CXCL10 polypeptide consists of an amino acid sequence of SEQ ID NO: 3 or SEQ ID NO: 4. In one embodiment, the polynucleotide encoding CXCL10 polypeptide comprises the sequence of SEQ ID NO: 7 or SEQ ID NO: 8. In one embodiment, the polynucleotide encoding CXCL10 polypeptide consists of the sequence of SEQ ID NO: 7 or SEQ ID NO: 8. In one embodiment, a cell comprising the polynucleotide encoding the CXCL9 polypeptide comprises the sequence of SEQ ID NO:5 or SEQ ID NO:6. In one embodiment, a cell comprising the polynucleotide encoding the CXCL10 polypeptide comprises the sequence of SEQ ID NO:7 or SEQ ID NO:8. In one embodiment, a cell comprising the polynucleotide encoding the CXCL9 polypeptide and the CXCL10 polypeptide comprises the sequence of SEQ ID NO:5 or SEQ ID NO:6, and SEQ ID NO:7 or SEQ ID NO:8.

### Immune Checkpoint Inhibition

Immune checkpoint inhibition, checkpoint inhibition, immune checkpoint inhibitor or checkpoint inhibitor refer similarly to a therapeutic agent that inhibits one or more of the immune checkpoints that suppresses T cell engagement. Non-limiting examples of checkpoints include CTLA-4, PD-1 and PD-L1 ; additional examples are provided below. Non limiting examples of immune checkpoint inhibitors include antibodies such as anti-CTLA-4, anti-PD-1, anti-PD-L1, and those described in more detail further below. Any one or more checkpoint inhibitor may be used with the CXCL9/10 as described herein in the practice of the invention.

In one non-limiting example, PD-1 is an immunoglobulin in the CD28 family. PD-1 is a type I transmembrane glycoprotein containing an extracellular Ig variable-type (V-type) domain involved in ligand binding and a cytoplasmic tail involved in intracellular signaling. Binding of PD-1 with PD-L1 (or the other ligand PD1-L2) induces the recruitment of SHP-1 and SHP-2 to PD-1, resulting in de-phosphorylation of CD3, PKCO and ZAP70 essential for T cell receptor (TCR) signaling, and down-regulation of T lymphocyte activation. Under healthy conditions, PD-L1 attenuates unwanted immune responses, such as autoimmunity.

Pembrolizumab is a humanized anti-PD-1 antibody used in cancer immunotherapy. Pembrolizumab is a highly selective humanized mAb designed to block the interaction between PD-1 and its ligands, programmed cell death ligand 1 (PD-L1) and programmed cell death ligand 2 (PD-L2). Pembrolizumab is an IgG4/kappa isotype with a stabilizing sequence alteration in the Fc region. The theoretical molecular weights of the heavy and light chains derived from the amino acid sequences, excluding glycosylation, are 49.4 kiloDaltons (KDa) and 23.7 KDa, respectively.

Clinical trials testing the safety and efficacy of pembrolizumab in treating NSCLC patients have led to the approval of the agent for this disease. The KEYNOTE-001 study revealed responses in approximately 20% of the patients with a modest side-effect profile. Importantly, patients with >50% PD-L1 baseline tumor staining experienced greater benefit from anti-PD-1 therapy than those with <50% tumor PD-L1 expression, with the ORR defined by Response Evaluation Criteria in Solid Tumors (RECIST) criteria of 45.2% in patients with >50% PD-L1 staining, versus 16.5% in patients with 1-49% PD-L1 staining and 10.7% in patients with <1% PD-L1 staining. In the KEYNOTE-010 phase II/III study, 2 mg/kg and 10 mg/kg q3w of pembrolizumab showed significant benefit over 75 mg/m2 q3w docetaxel in randomized, stage IV pre-treated patients with >1% PD-L1 staining. In the KEYNOTE-024 study, 200 mg of pembrolizumab showed significant benefit over investigators' choice of standard of care chemotherapy in treatment-naïve patients with >50% PD-L1 staining. Despite robust and durable responses to anti-PD-1 therapy in a subgroup of NSCLC patients, most patients do not respond to PD-1 checkpoint inhibitors as single agents (12). Therefore, rational and effective combination strategies with PD-1 inhibitors are needed to enhance the efficacy of the anti-PD1 therapy in advanced NSCLC patients.

Antibodies to PD-1 have been described in U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; and 8,217,149.

It is contemplated that any known anti-PD-1 antibody can be used in the present methods. In various embodiments, the anti-PD-1 antibody inhibits or blocks binding of the PD-1 receptor to one or both of its ligands, PD-L1 and PD-L2. In exemplary aspects, the monoclonal antibody that specifically binds to PD-1 is Nivolumab (BMS936558; Bristol Meyers Squibb), Pembrolizumab (MK-3475; Merck), Pidilizumab (CT-011; CureTech), Lambrolizumab, BMS-936559, Atezolizumab, or AMP-224 (GSK/Amplimmune), AMP224 (MedImmune); AUNP12 (Dr. Reddy's Laboratories Ltd.); BGB108 (BeiGene); MCLA134 (Merus BV); MEDI0680 (Medlmmune); PDR001 (Novartis); REGN2810 (Regeneron/Sanofi); SHR1210 (Jiangsu Hengrui Medicine/Incyte); STIA110X (Sorrento); STIA1110 (Sorrento); TSR042 (AnaptysBio/Tesaro).

In exemplary aspects, the monoclonal antibody that specifically binds to PD-L1 is BMS-936559 (BMS/Ono), MPDL3280A (Roche/Genentech), or MEDI-4736 (Medlmmune), MSB0010718C (Merck/Serono), ALN-PDL (Alnylam); BGBA317 (BeiGene); KDO33 (Kadmon Corp.); KY1003 (Kymab Ltd.); STIA100X (Sorrento); STIA1010 (Sorrento); STIA1011 (Sorrento); STIA1012 (Sorrento); and STIA1014 (Sorrento).

For studies using anti-PD-1 in the mouse, a mouse PD-1 specific antibody is used, such as but not limited to monoclonal antibody clone RMP-1-14, catalog no. BP0146, from Bio X Cell, Lebanon N.H. Studies in other animal models can use the appropriate species specific antibody.

Other immune checkpoint inhibitors are embraced herein, and the invention is not limited to any particular immune checkpoint inhibitor co-administered with CXCL9/10 as described herein.

As used herein “Programmed cell death protein 1” or “PD-1” refers to a cell surface receptor involved in immune checkpoint blockade mediated by binding to two ligands, PD-L1 and PD-L2. PD-1 binding to its ligands has been shown to reduce T-cell proliferation, cytokine production, and cytotoxic activity.

Cytotoxic T-lymphocyte antigen 4 (CTLA-4)(CD152) is a well-known costimulatory molecule involved in the B7-1/B7-2 costimulatory pathway of T cell activation. CTLA-4 is expressed on the surface of helper T cells and transmits an inhibitory signal to T cells (See e.g., Krummel et al., J. Exp. Med. 182 (2): 459-65, 1995). Antibodies that bind CTLA-4 include ipilimumab and tremilimumab.

An “antigen presenting cell” (APC) is a cell that is capable of activating T cells, and includes, but is not limited to, monocytes/macrophages, B cells and dendritic cells (DCs). The term “dendritic cell” or “DC” refers to any member of a diverse population of morphologically similar cell types found in lymphoid or non-lymphoid tissues. These cells are characterized by their distinctive morphology, high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes.

The term “therapeutically effective amount” is used herein to indicate the amount of target-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

The terms “cancer”, “cancerous”, or “malignant” refer to or describe the physiological condition in mammals that is typically characterized by unregulated cell growth. Exemplary cancers contemplated herein are described more fully in the Detailed Description.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “pharmaceutical composition” refers to a composition suitable for administration to a subject animal, including humans and mammals. A pharmaceutical composition comprises a pharmacologically effective amount of a virus or antigenic composition of the invention and also comprises a pharmaceutically acceptable carrier. A pharmaceutical composition encompasses a composition comprising the active ingredient(s), and the inert ingredient(s) that make up the pharmaceutically acceptable carrier, as well as any product which results, directly or indirectly, from combination, complexation or aggregation of any two or more of the ingredients. Accordingly, the pharmaceutical compositions of the present invention encompass any composition made by admixing a compound or conjugate of the present invention and a pharmaceutically acceptable carrier.

### CXCL9/10 Modified Dendritic Cells

### Vectors

A method for delivery of a CXCL9/10 expression construct involves the use of an expression vector. In one embodiment, for the studies described herein, DC were transduced with a lentiviral construct expressing murine CXCL9 and, separately, other DC were transduced with a lentiviral construct expressing murine CXCL10. In one embodiment a vector encoding both CXCL9 and CXCL10 can be provided on the same vector, or separate vectors encoding each CXCL can be introduced into the same cells. However, the invention is no so limiting.

In one embodiment, a lentiviral vector may be used for improved stability and better expression. Alternatively, other vectors can be used to express CXCL9 or CXCL10 or both.

The nucleotide sequence for CXCL9 may be obtained based on the sequence provided in SEQ ID NO:2. PCR products of CXCL9 may be generated and “sticky ends” generated through restriction digestion. These products are then ligated onto a lentiviral vector. This vector is then transfected with helper vectors onto 293T cells to generate lentivirus containing CXCL9 protein expression which were subsequently used to infect dendritic cells for delivery into the tumor. CXCL10-DCs may be generated using a similar process. Alternatively, other viral vectors can be used.

Exemplary vectors include viral vectors, liposomes or plasmid vector, as well as other gene delivery vectors. Viral vectors include adenovirus, an adeno-associated virus, a lentivirus, a retrovirus, a vaccinia virus, modified Ankara virus, and vesicular stomatitis virus.

Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct in host cells with complementary packaging functions and (b) to ultimately express a heterologous gene of interest that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences (Grunhaus and Horwitz, 1992). In contrast to retrovirus, the adenoviral infection of host cells does not result in chromosomal integration because wild-type adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity.

In various embodiments, the vector is a replication deficient adenoviral vector.

In various embodiments, the adeno-associated virus (AAV) is selected from the group consisting of AAV1, AAV2, AAV3, AAV4, AAV5, AAV6, AAV7, AAV8 or combinations thereof.

In one example as described in detail below, DC were transduced with a lentiviral construct expressing murine CXCL9 and CXCL10.

For the uses herein, DC may be obtained from the patient by leukapheresis or other method to collect dendritic cells from the patient's blood. The DC will then be cultured and transduced with the vector comprising the polynucleotide encoding CXCL9 or CXCL10. In other embodiments, DC are from a donor or cell line. In one embodiment the donor or cell line DCs are HLA matched to the recipient. In one embodiment the DCs are partially HPA matched.

### Methods of Use

Exemplary conditions or disorders that can be treated with the proposed combination therapy include cancers, such as esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16; 16)(p13.1; q22); CBFB-MYH11; adult acute myeloid leukemia with t(8; 21)(q22; q22); RUNX1-RUNX1T1; adult acute myeloid leukemia with t(9; 11)(p22; q23); MLLT3-MLL; adult acute promyelocytic leukemia with t(15; 17)(q22; q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteo sarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

In various embodiments, the cancer is lung cancer. In various embodiments, the lung cancer is non-small cell lung carcinoma (NSCLC). In various embodiments, the lung cancer is stage IV NSCLC expressing PD-L1 in less than 50% of cells.

In various embodiments, the NSCLC or other solid tumor is a squamous cell or non-squamous cell tumor. In various embodiments, the subject has a low tumor mutational burden. In various embodiments, the subject has a high tumor mutational burden. Tumor mutational burden may be monitored by diagnostic assay, e.g., from FoundationOne (Cambridge, Mass.), such as FoundationOne CDx™, FoundationOne®, FoundationAct®, or FoundationOne®Heme.

In various embodiments, the patient has a NSCLC tumor accessible by CT-guided intervention or bronchoscopy, and the patient is naïve to systemic treatment for NSCLC. In various embodiments, the CXCL9/10-DC is administered via CT-guided or bronchoscopic IT injection.

As described herein, certain cancers are characterized by having a high mutational burden. In various embodiments, a method of treating cancer or a solid tumor having a high mutational burden in a subject is provided comprising a. administering to the subject (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) any combination thereof; and (b) administering to the subject an immune checkpoint inhibitor.

In various embodiments, a method of treating a cancer or a solid tumor in a subject is provided comprising the steps of a. identifying the presence of a high mutational burden in the tumor of the subject; b. administering to the subject having a high mutational burden tumor (i) a CXCL9 polypeptide, CXCL10 polypeptide, or the combination thereof, (ii) a polynucleotide encoding the CXCL9 polypeptide, CXCL10 polypeptide, or the combination thereof, (iii) a cell comprising the polynucleotide encoding the CXCL9 polypeptide, CXCL10 polypeptide, or the combination thereof, or (iv) any combination thereof; and (b) administering to the subject an immune checkpoint inhibitor.

In one embodiment of the foregoing methods, the high mutational burden is determined by a biopsy of the tumor. In one embodiment, tumor-associated neoantigens are determined. In one embodiment, the efficacy of combination therapy is followed by elucidation of the neoantigen landscape of the tumor. In one embodiment, the tumor comprises a mutation selected from KRAS, TP53 (KP) or STK11/LKB1, or any combination thereof. In one embodiment, the tumor has intratumoral heterogeneity. In one embodiment, the tumor mutational burden is determined by diagnostic assay selected from FoundationOne CDx™, FoundationOne®, FoundationAct®, and FoundationOne®Heme.

In one embodiment, the high mutational burden tumor does not have an activating mutation in the epidermal growth factor receptor or an anaplastic lymphoma kinase gene (ALK) fusion. In one embodiment, the somatic mutational load and tumor-associated neoantigens before, during and after treatment are used to initiate, prescribe and monitor therapy.

In one embodiment, a method for treating cancer or reducing the reoccurrence of a high mutational burden cancer in a subject in need thereof comprising administering an effective amount of a combination therapy comprising a) dendritic cells comprising an CXCL9/10 construct on days 0, 21, and 42, and b) an effective amount of anti-PD-1 antibody every three weeks starting on day 0.

In various embodiments thereof, the tumor does not have an activating mutation in the epidermal growth factor receptor or an anaplastic lymphoma kinase gene (ALK) fusion.

In various embodiments thereof, the somatic mutational load and tumor-associated neoantigens before, during and after treatment are used to initiate, prescribe and monitor therapy.

In some embodiments, in the cancer treated with the present method, less than 50% of tumor cells express the PD-L1 protein on their surface. In various embodiments, the cancers have greater than 50% PD-L1 staining on their cell surface, and therefore greater than 50% of tumor cells express the PD-L1 protein on their surface.

It is further contemplated that the present methods are useful in subjects treated with first line pembrolizumab plus chemotherapy, or, alternatively, in subjects that fail initial therapy with this combination.

In some embodiments, cancers that can be treated with the present methods include metastatic NSCLC and other solid tumors as described herein.

In various embodiment, the methods of the invention can be used to treat patients with low mutational burden tumors.

It is contemplated that the methods herein reduce tumor size or tumor burden in the subject, and/or reduce metastasis in the subject. In various embodiments, tumor size or tumor volume in the subject is decreased by about 25-50%, about 40-70% or about 50-90% or more. In various embodiments, the methods reduce the tumor size or tumor volume by 10%, 20%, 30%, or more. In various embodiments, the methods reduce tumor size or tumor volume by 10%, 15%, 20%, 25%, 30%, 35%, 40%, 45%, 50%, 55%, 60%, 65%, 70%, 75%, 80%, 85%, 90%, 95% or 100%.

It is contemplated that the methods herein reduce tumor burden, and also reduce or prevent the recurrence of tumors once the cancer has gone into remission.

It is also contemplated that administration of the CXCL9/10-DC increases CD8 T cell infiltration into a tumor. In various embodiments, the CD8 cells are increased by 2-fold or more in the treated subject compared to a subject not receiving combination therapy. It is provided that the CXCL9/10-DC increases PD-L1 expression in a tumor.

In various embodiments, the one or both CXCL9 and CXCL10 polypeptides, one or both CXCL9 and CXCL10 polynucleotides, or antigen presenting cells such as dendritic cells expressing CXCL9, CXCL10, or both, are administered intratumorally, intravenously, intra-arterially, intraperitoneally, intranasally, intramuscularly, intradermally or subcutaneously, or via CT-guided or bronchoscopic IT injection. In various embodiments, the checkpoint inhibitor is administered intravenously.

The route of administration of the CXCL9 polypeptide, CXCL10 polypeptide, or the combination thereof, polynucleotide or APC such dendritic cells, and of the checkpoint inhibitor will vary depending on the desired outcome. Generally, for initiation of an immune response, injection of the agent at or near the desired site of inflammation or response is utilized. Alternatively, other routes of administration may be warranted depending upon the disease condition. That is, for suppression of neoplastic or tumor growth, injection of the pharmaceutical composition at or near the tumor site is preferred.

As noted herein, the CXCL9/10 may comprise CXCL9, CXCL10, or both. The form of administration of one or both may be in the form of a polypeptide, a polynucleotide, or a cell comprising the polypeptide. Both the CXCL and the form of administration are independently selected for each CXCL. Thus, in some embodiments, CXCL9 may be delivered as a CXCL9 polypeptide, CXCL9 encoding polynucleotide or cell comprising the polynucleotide encoding CXCL9, or any combination thereof. Thus, in some embodiments, CXCL10 may be delivered as a CXCL10 polypeptide, CXCL10 encoding polynucleotide or cell comprising the polynucleotide encoding CXCL10, or any combination thereof. In some embodiments, both CXCL9 and CXCL10 may be administered; CXCL9 may be delivered as a CXCL9 polypeptide and CXCL10 delivered as CXCL10 polypeptide; or as CXCL9 encoding polynucleotide and CXCL10 encoding polynucleotide; or cell comprising the polynucleotide encoding CXCL9 and the polynucleotide encoding CXCL10, or any combination thereof. In other embodiment wherein both CXCL9 and CXCL10 are delivered, each may be independently delivered as a polypeptide, polynucleotide or cell encoding the polynucleotide, or any combination thereof. By way of non-limiting example, the CXCL9 may be delivered as a polypeptide and the CXCL10 as a polynucleotide. In another example, CXCL9 may be delivered as a polypeptide and CXCL10 delivered in a cell comprising the CXCL10 polynucleotide. In another example, the CXCL9 may be delivered by a cell comprising the CXCL9 polynucleotide, and the CXCL10 as a polypeptide. In another example, CXCL9 may be delivered as a polynucleotide and CXCL10 delivered in a cell comprising the CXCL10 polynucleotide. Thus, any combination of one or both CXCLs and one or more methods of delivery are embodied herein. Of course, an immune checkpoint inhibitor is also delivered. As noted herein, the dose level, dosing regimen such as frequency and duration of dosing, of the CXCL9/10 and the immune checkpoint inhibitor (including one or more immune checkpoint inhibitors) is provided herein.

As noted herein, a polynucleotide encoding human CXCL9 may comprise SEQ ID NO:5 or consist of SEQ ID NO:5. As noted herein, a polynucleotide encoding murine CXCL9 may comprise SEQ ID NO:6 or consist of SEQ ID NO:6. As noted herein, a polynucleotide encoding human CXCL10 may comprise SEQ ID NO:7 or consist of SEQ ID NO:7. As noted herein, a polynucleotide encoding murine CXCL10 may comprise SEQ ID NO:8 or consist of SEQ ID NO:8.

As noted herein, a cell comprising the polynucleotide encoding the CXCL9 polypeptide, CXCL10 polypeptide, or the combination thereof may comprise a polynucleotide encoding human CXCL9 comprising SEQ ID NO:5 or consisting of SEQ ID NO:5. As noted herein, a cell comprising a polynucleotide encoding murine CXCL9 may comprise SEQ ID NO:6 or consist of SEQ ID NO:6. As noted herein, a cell comprising a polynucleotide encoding human CXCL10 may comprise SEQ ID NO:7 or consist of SEQ ID NO:7. As noted herein, a cell comprising a polynucleotide encoding murine CXCL10 may comprise SEQ ID NO:8 or consist of SEQ ID NO:8. As noted herein, a cell comprising a polynucleotide encoding human CXCL9 and human CXCL10 may comprise SEQ ID NO:5 or consist of SEQ ID NO:5, and may comprise SEQ ID NO:7 or consist of SEQ ID NO:7. As noted herein, a cell comprising a polynucleotide encoding murine CXCL9 and murine CXCL10 may comprise SEQ ID NO:6 or consist of SEQ ID NO:6, and may comprise SEQ ID NO:8 or consist of SEQ ID NO:8. In some embodiments, a cell may comprise both a human and murine chemokine.

In one embodiment, the form of CXCL9 and independently the form of CXCL10, may be delivered intratumorally (IT).

It should be noted that despite the use of the term combination, the CXCL9/10 and checkpoint inhibitor do not need to be administered at the same time of by the same route of administration or in the same formulation. Each may have a different dosing schedule; in one embodiment, the dosing schedules are temporally overlapping; in one embodiment one is started before the other; in one embodiment one ends before the other; in one embodiment the course of therapy of one agent ends before the other begins (wherein the anti-tumor efficacy of both agents is greater than either alone; i.e., is synergistic). Combination refers to the concurrent use of both a CXCL9/10 and a checkpoint inhibitor to achieve the anti-tumor activity described herein. In one embodiment, the anti-tumor effect of the combination of the CXCL9/10 and immune checkpoint inhibitor is greater than either agent individually; in one embodiment the effect is greater than the combination of the effect of each agent individually; in one embodiment, the combination is synergistic.

Examples of other routes of systemic administration include parenteral, e.g., intravenous, intradermal, subcutaneous, oral (e.g., inhalation) transdermal (topical), transmucosal, and rectal administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

In one embodiment, the pharmaceutical composition can be delivered via slow release formulation or matrix comprising CXCL9 polypeptide, CXCL10 polypeptide, or the combination thereof or DNA constructs suitable for expression of CXCL9/10 into or around a site within the body. In this manner, a transient lymph node can be created at a desired implant location to attract dendritic cells and T cells initiating an immune response.

The selection of the one or more immune checkpoint inhibitors to be administered with the CXCL9/10 may be guided as described herein. While the examples herein refer to anti-PD1 antibody, the invention is not so limited and is generally directed to the combination of CXCL9/10 with any one or more immune checkpoint inhibitor.

In the example of anti-PD-1, it is contemplated that the anti-PD-1 antibody is administered every three weeks starting on day 0. In various embodiments, the anti-PD-1 antibody is pembrolizumab administered at a dose of 200 mg every three weeks.

In various embodiments, each of the CXCL9-DC and CXCL10-DC are administered in a dose from 5×10⁶ cells/injection to 3×10⁷ cells/injection, e.g., 5×10⁶, 1×10⁷, or 3×10⁷ cells/injection. It is contemplated that the dendritic cells comprising a vector-CXCL9/10, such as a lentiviral construct (CXCL9/10-DC), are administered at 3-week intervals, e.g., on days 0, 21, and 42. As noted, these are merely exemplary dosing regiments and the skilled artisan carrying out the invention will readily determine the optimal dosing regimen for the particular subject, the tumor stage, etc.

It is further contemplated that other adjunct therapies may be administered, where appropriate. For example, the patient may also be administered surgical therapy, chemotherapy, a cytotoxic agent, photodynamic therapy or radiation therapy where appropriate.

A wide variety of chemotherapeutic agents may be used in combination with the combination therapy of the present invention. These can be, for example, agents that directly cross-link DNA, agents that intercalate into DNA, and agents that lead to chromosomal and mitotic aberrations by affecting nucleic acid synthesis. A variety of chemotherapeutic agents are intended to be of use in the combined treatment methods disclosed herein. Chemotherapeutic agents contemplated as exemplary include, e.g., etoposide (VP-16), adriamycin, 5-fluorouracil (5FU), camptothecin, actinomycin-D, mitomycin C, cisplatin (CDDP) and even hydrogen peroxide.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/in2 for 5 days every three weeks for a total of three courses. Cisplatin is not absorbed orally and must therefore be delivered via injection intravenously, subcutaneously, intratumorally or intraperitoneally.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/in2 at 21-day intervals for adriamycin, to 35-50 mg/in2 for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Plant alkaloids such as TAXOL are also contemplated for use in certain aspects of the present invention. TAXOL is an experimental antimitotic agent, isolated from the bark of the ash tree, Taxus brevifolia. It binds to tubulin (at a site distinct from that used by the vinca alkaloids) and promotes the assembly of microtubules. TAXOL is currently being evaluated clinically; it has activity against malignant melanoma and carcinoma of the ovary. Maximal doses are 30 mg/m2 per day for 5 days or 210 to 250 mg/m 2 given once every 3 weeks. Of course, all of these dosages are exemplary, and any dosage in-between these points is also expected to be of use in the invention.

The foregoing exemplary chemotherapeutic agents that are useful in connection with combined therapy are not intended to be limiting and use of such agent or agents will be guided by the skilled practitioner. Each of the agents listed therein are exemplary and by no means limiting. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLES

### Example 1. Newly Established Genetically Engineered Murine Model (GEMM) of Lung Cancer Bearing Varying Mutational Loads Recapitulate Clinical Response to Anti-PD-1

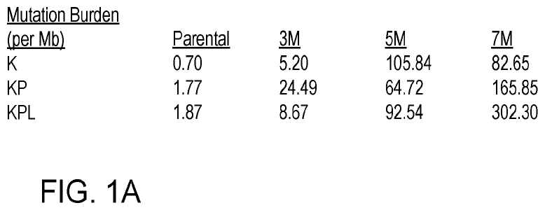

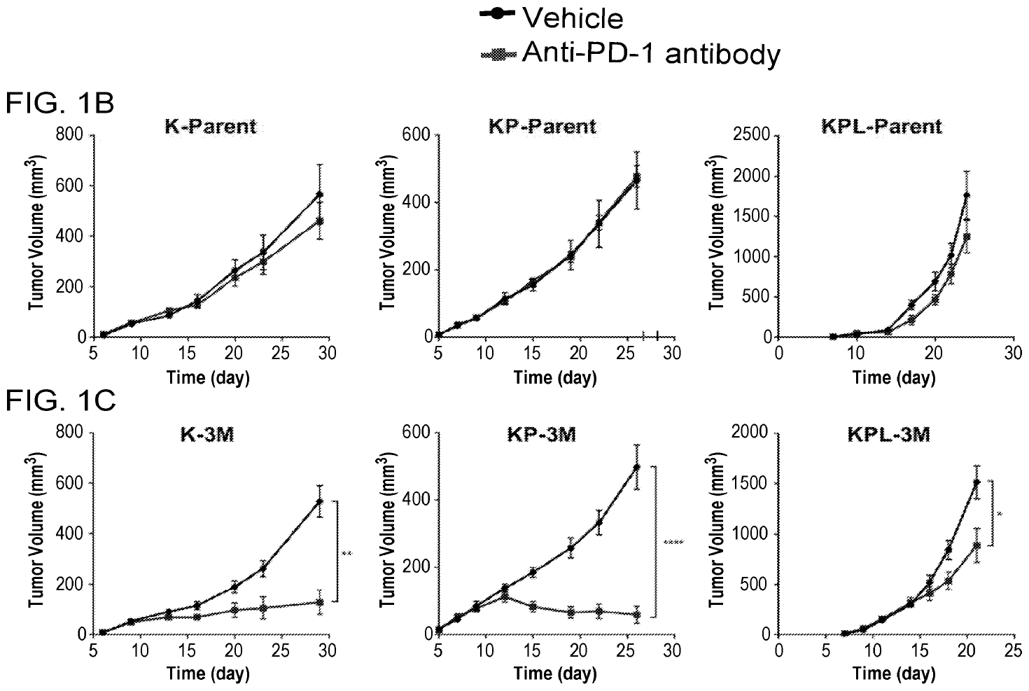

Although conditional GEMMs of NSCLC bear common driver mutations of the disease, recent studies reveal that these GEMMs possess low tumor mutational burden (TMB). To recapitulate the mutational landscape of human NSCLC, we established novel GEMMs with increased TMB by in vitro exposure of Kras^{G12D} (K), Kras^{G12D}P53^{-/-} (KP) and Kras^{G12D}P53^{-/-} Lkb1^{-/-} (KPL) cells to carcinogen methyl-nitrosourea (MNU) for 30 min each time for 3, 5 or 7 times (designated as 3M, 5M, 7M, respectively). MNU is a potent alkylating agent that targets the nitrogen and oxygen atoms of nucleotides, resulting in transitions with similar numbers of G>A and C>T substitutions. Whole exome sequencing (WES) of these mutant cell lines revealed significant increases in mutational loads (FIG. 1A), as well as intratumoral heterogeneity. While tumors from all three parental cell lines are resistant to anti-PD-1 therapy (FIG. 1B), PD-1 blockade elicited potent efficacy in K-3M and KP-3M models (FIG. 1C). In contrast, limited anti-PD-1 efficacy was observed in the KPL-3M tumors (FIG. 1D). Recent studies revealed that ORR to anti-PD-1 monotherapy differed significantly among KL (7.4%), KP (35.7%), and K (28.6%) subgroups of human KRAS-mutant LUAC, identifying LKB1 loss of function mutation as a major driver of primary resistance to anti-PD-1. Our results recapitulate clinical responses to anti-PD-1 therapy in NSCLC, and highlight the utility of these novel GEMMs in studying molecular mechanisms of responses to immunotherapy.

Detailed description of FIG. 1A, FIG. 1B, FIG. 1C and FIG. 1D. Newly established GEMMs of lung cancer bearing varying mutational loads recapitulate clinical response to anti-PD-1. FIG. 1A) WES was performed with genomic DNA from KrasG12D (K), KrasG12DP53^{-/-} (KP) and KrasG12DP53^{-/-}Lkb1^{-/-} (KPL) parental and mutant cells (3M, 5M, 7M). DNA from mouse tail was included as normal tissue control. Numbers represent mutation burden as single-nucleotide variants (SNVs) per Mb. FIG. 1B) Post-tumor inoculation [K-Parent (2×106) cells SC in 129/E mice; KP-Parent (8×10⁵) or KPL-Parent (7.5×10⁴) cells SC in FVB mice)], mice bearing <50 mm3 tumors (˜day 5-7) were treated with i) vehicle, ii) Anti-PD-1 antibody (200 μg/dose IP every 3 days for 4 doses), and tumor growth was measured with caliper. FIG. 1C) Same as FIG. 1B except 3M cells were utilized [K-3M (2×10⁶) cells in 129/E mice; KP-3M (2.2×10⁶) or KPL-3M (1×10⁵) cells in FVB mice. P values were determined by non-paired t-test. n.s., not significant; *, P<0.05; **, P<0.005; ***, P<0.0005; ****, P<0.0001.

### Example 2. Distinct TME Immune Phenotypes Occur in GEMM Subtypes

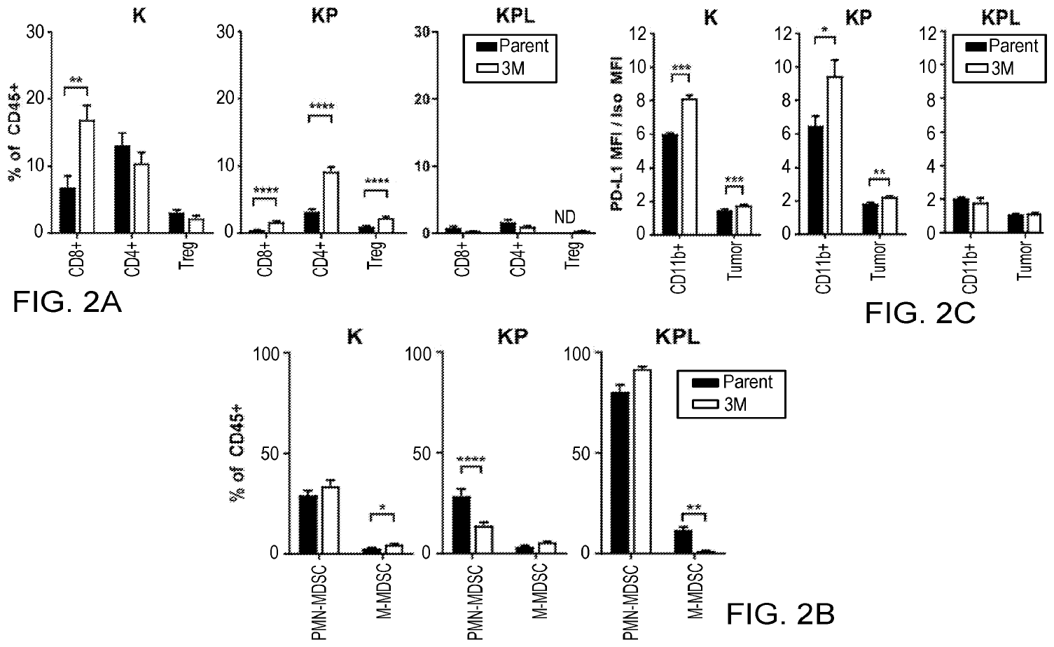

Responses to anti-PD-1 therapy require pre-existing tumor-specific T cell immunity that is restrained by PD-L1/PD-1-mediated suppression. Consistent with the observation that KRAS-driven lung cancers with LKB1-inactivating mutations are resistant to anti-PD-1 therapy, our immune profiling of the tumor microenvironment (TME) of the GEMMs by flow cytometry revealed significant infiltration of T lymphocytes in K-3M and KP-3M tumors but a lack of CD4⁺ and CD8⁺ T cell infiltration in KPL-P and KPL-3M tumors, indicative of a state of suppressed T cell immunity (FIG. 2A). In contrast, we observed a predominance of myeloid derived suppressor cells (MDSCs) within the TME of KPL-P and KPL-3M (FIG. 2B), consistent with the previous studies. As anticipated, the profound state of immunosuppression in KPL-P and KPL-3M tumors with low T lymphocyte infiltration correlates with low PD-L1 expression, which is associated with primary resistance to anti-PD-1 monotherapy (FIG. 2C). We have found increased levels of chemoattractant cytokines in LKB1-null KPL-3M tumors, including CXCL1, 2, 3, 5, 7 and IL-6, which may contribute to the recruitment of MDSCs and the profound state of T cell suppression within the TME.

Detailed description of FIG. 2A, FIG. 2B and FIG. 2C. Distinct TME immune phenotypes in three genetic subtypes of GEMMs. FIG. 2A) On day 14-16 post-tumor inoculation (2×10⁶ K-Parent and K-3M delivered SC in 129/E mice; KP-Parent (8×10⁵) and KP-3M (2.2×10⁶) cells in FVB mice; or KPL-Parent (1×10⁵) and KPL-3M (1.5×10⁵) cells in FVB mice), tumors were harvested and analyzed by FACS using T cell markers. Treg is defined as CD45+CD4⁺FoxP3⁺. FIG. 2B) Same as FIG. 2A except that MDSC markers were utilized. Polymorphonuclear (PMN)-MDSC is defined as CD45⁺CD11b⁺Ly6G⁺Ly6C^{lo}. Monocytic (M)-MDSC is defined as CD45⁺CD11b⁺Ly6G⁻Ly6C^{hi}. FIG. 2C) Same as FIG. 2A except that mean fluorescence intensity (MFI) of PD-L1 is plotted. P values were determined by non-paired t-test. *, P<0.05; **, P<0.005; ***, P<0.0005; ****, P<0.0001. ND, not determined.

### Example 3. Enhanced Efficacy of Anti-PD-1 by IT Administration of CXCL9/10-DC in a Murine Lung Cancer Model

For the studies described herein, DC were transduced with a lentiviral construct expressing murine CXCL9 and, separately, other DC were transduced with a lentiviral construct expressing murine CXCL10. The nucleotide sequence for CXCL9 (SEQ ID NO: 6) was obtained based on the sequence provided in SEQ ID NO:2. PCR products of CXCL9 were generated and “sticky ends” generated through restriction digestion. These products were then ligated onto a lentiviral vector. This vector was then transfected with helper vectors onto 293T cells to generate lentivirus containing CXCL9 protein expression which were subsequently used to infect dendritic cells for delivery into the tumor. CXCL10-DCs were generated using a similar process using the nucleotide sequence (SEQ ID NO:8) based on SEQ ID NO:4. These DCs were referred to herein as CXCL9/10-DC (or vector-CXCL9/10-DC). As noted herein, for each DC population, about 1×10\hat{ }6 cells produced 10 ng of the respective polypeptide in 24 hours. About 1×10\hat{ }6 cells expressing CXCL9 and about 1×10\hat{ }6 cells expressing CXCL10 (i.e., a total of 2×10\hat{ }6 cells) were administered to animals.

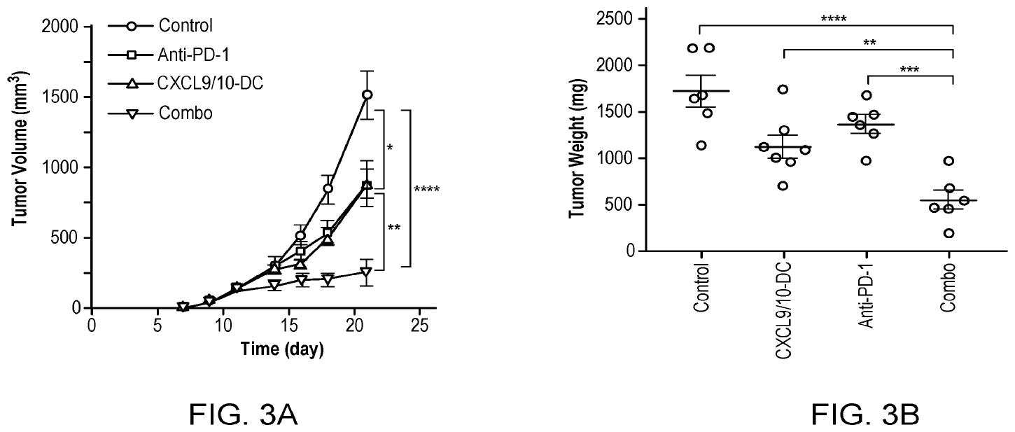

We performed studies to evaluate the efficacy of CXCL9/10-DC and anti-PD-1 (anti-moue PD-1 (CD279), clone RMP-1-14, catalog no. BP0146, from Bio X Cell, Lebanon N.H.) combination in a KPL-3M GEMM of lung cancer. Consistent with the observed immunosuppressive TME (FIG. 2A-C), anti-PD-1 alone showed limited efficacy (FIG. 3A). Importantly, CXCL9/10-DC significantly potentiated the anti-tumor effect of PD-1 blockade. Tumor weights on the day of euthanasia were consistent with the tumor volume measurements (FIG. 3B). These data show that CXCL9/10-DC in combination with PD-1 elicits profound anti-tumor effect.

Brief description of FIG. 3A and FIG. 3B. The combination of CXCL9/10-DC and anti-PD-1 outperforms monotherapies. FIG. 3A) IT CXCL9/10-DC (DC transduced with a lentiviral construct expressing murine CXCL9 and CXCL10 and anti-PD-1 combination in murine KPL-3M model. FVB mice were SC inoculated with 1×10⁵ KPL-3M cells. On day 7, mice bearing <25 mm³ tumors were treated with a) vehicle control; b) IT CXCL9/10-DC (10⁶ CXC9/10-DC/dose on day 7, 11, 15); c) IP anti-PD-1 (200m/dose on day 7, 9, 11, 13, 15); d) combination of IT CXCL9/10 and IP anti-PD-1 at the same time points as above. Tumor volume as measured by calipers was recorded. FIG. 3B) Same as in FIG. 3A except that tumor weight at the end of the study was presented. P values were determined by non-paired t-test; *, P<0.05; **, P<0.005; ***, P<0.0005; ****, P<0.00005.

### Example 4. CXCL9/10 Gene Expression Levels Correlate with DC and CD8⁺ T Cell Infiltration

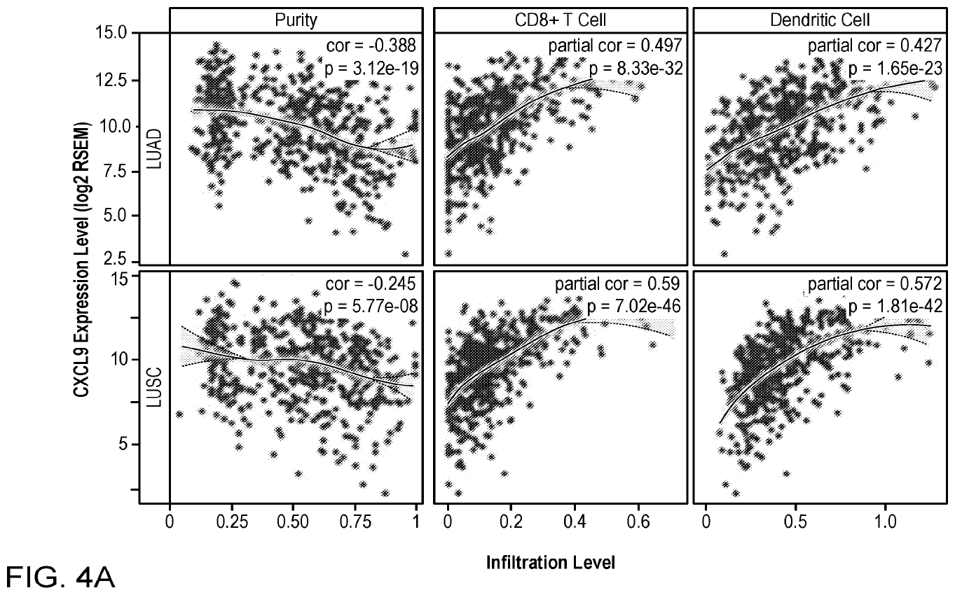

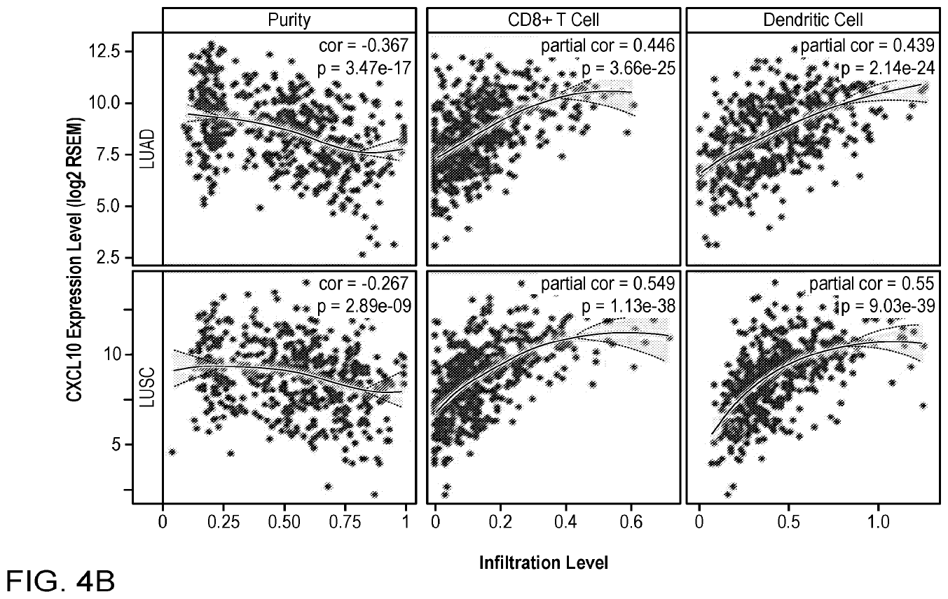

To determine the association between CXCL9/10 expression and immune infiltration, we analyzed TCGA NSCLC data including both lung adenocarcinoma (LUAD) and lung squamous carcinoma (LUSC). TIMER was used to estimate the immune cell population based on gene expression levels. Both CXCL9 (FIG. 4A) and CXCL10 (FIG. 4B) gene expression levels significantly correlated with the estimated infiltrating CD8⁺ T cell and DC populations. CXCL9/10 expression does not correlate with the tumor content.

Detailed description of FIG. 4A and FIG. 4B. Correlation between CXCL9/10 gene expression to CD8⁺ T cells and DCs in TCGA data. CXCL9 (FIG. 4A) and CXCL10 (FIG. 4B) expression levels were plotted against Purity (Tumor content), CD8⁺ T cell and DC populations were estimated for LUAD (top) and LUSC (bottom) for NSCLC. The level of tumor content was not correlated with CXCL9 or CXCL10 levels, while CD8⁺ T cell and DC showed significant correlation.

### Example 5. Intratumoral (IT) Administration of CXCL9/10-DC Enhances the Efficacy of Anti-PD-1 in the KPL-3M Model

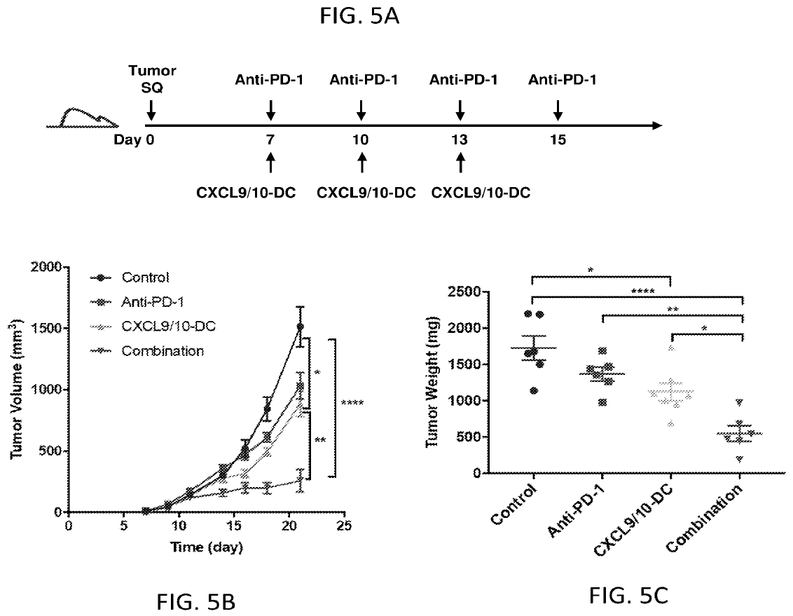

In a similar study design as that described in Example 3 (except for the dosing regimen), the efficacy of CXCL9/10-DC and anti-PD-1 combination therapy was evaluated in the KPL-3M murine model. The study design is shown in FIG. 5A. Tumors were implanted subcutaneously on Day 0. On Days 7, 10, 13 and 15, animals were administered CXCL9/10 intratumorally and anti-PD-1. As shown in FIG. 5B, anti-PD-1 or CXCL9/10 monotherapy showed limited efficacy. Importantly, CXCL9/10-DC significantly potentiated the antitumor effect of PD-1 blockade (FIG. 5B and FIG. 5C) with regard to tumor growth and tumor volume, respectively.

To evaluate the synergy of the combination of CXCL9/10 and anti-PD1, doubling times of the tumor volume curves were calculated, compared to the control group doubling time, and the change and percent change in doubling time among the groups compared. A longer doubling time indicates a reduction in tumor growth. As shown in the table below, the decrease in doubling time of the combination treatment of CXCL9/10-DC and anti-PD-1, is 2.7-fold greater than the sum of the decrease in doubling times of the individual treatments.

| | | | Sum of | Combination |
|---|---|---|---|---|
| | | | anti-PD-1 and | anti-PD-1 and |
| Tumor volume | | CXCL9/ | CXCL9/ | CXCL9/ |
| (doubling time) | Anti-PD-1 | 10-DC | 10-DC | 10-DC |
| Doubling time | 3.914 | 3.494 | — | 6.411 |
| (min) | | | | |
| Control = 3.079 | | | | |
| Change in | −0.84 | −0.42 | −1.26 | −3.33 |
| doubling time | | | | |
| from control | | | | |
| Percent change | 27% | 13% | 40% | 108% |
| in doubling | decrease | decrease | decrease | decrease |
| time from | | | | |
| control | | | | |

In a similar analysis, the change in tumor weight data are shown in the table below. The decrease in tumor weight of the combination treatment of CXCL9/10-DC and anti-PD-1, is 24% greater than the sum of the decrease in tumor weights of the individual treatments.

| | | | Sum of anti-PD-1 | Combination |
|---|---|---|---|---|
| | Anti- | CXCL9/ | and CXCL9/ | anti-PD-1 and |
| Tumor weight | PD-1 | 10-DC | 10-DC | CXCL9/10-DC |
| Tumor weight | 1127 | 1368 | — | 550 |
| (mg) | | | | |
| Control = 1727 | | | | |
| Change in | −600 | −358 | −958 | −1226 |
| average tumor | | | | |
| weight from | | | | |
| control | | | | |
| Percent change | 34% | 21% | 55% | 68% |
| in tumor weight | decrease | decrease | decrease | decrease |
| from control | | | | |

Detailed description of FIG. 5A, FIG. 5B and FIG. 5C. IT CXCL9/10-DC potentiates anti-PD-1 efficacy in the KPL-3M model. FIG. 5A) Schematic diagram of the study. FIG. 5B) FVB mice were SC inoculated with 1.5×10\hat{ }5 KPL-3M cells. On day 7, mice bearing <50 mm3 tumors were treated with a) vehicle; b) IP anti-PD-1 (200m/dose on days 7, 10, 13, 15); c) IT CXCL9/10-DC (10\hat{ }6 cells each/dose on days 7, 10, 13); d) combination of b) and c). Tumor volume was recorded. FIG. 5C) Tumor weights at the time of necropsy. P values were determined by non-paired t-test. *, P<0.05; **, P<0.005; ****, P<0.0001.

### Example 6. Intratumoral (IT) CXCL9/10-DC Promotes T Cell Infiltration and Reduces Immunosuppressive MDSC in the TME in the KPL-3M Model

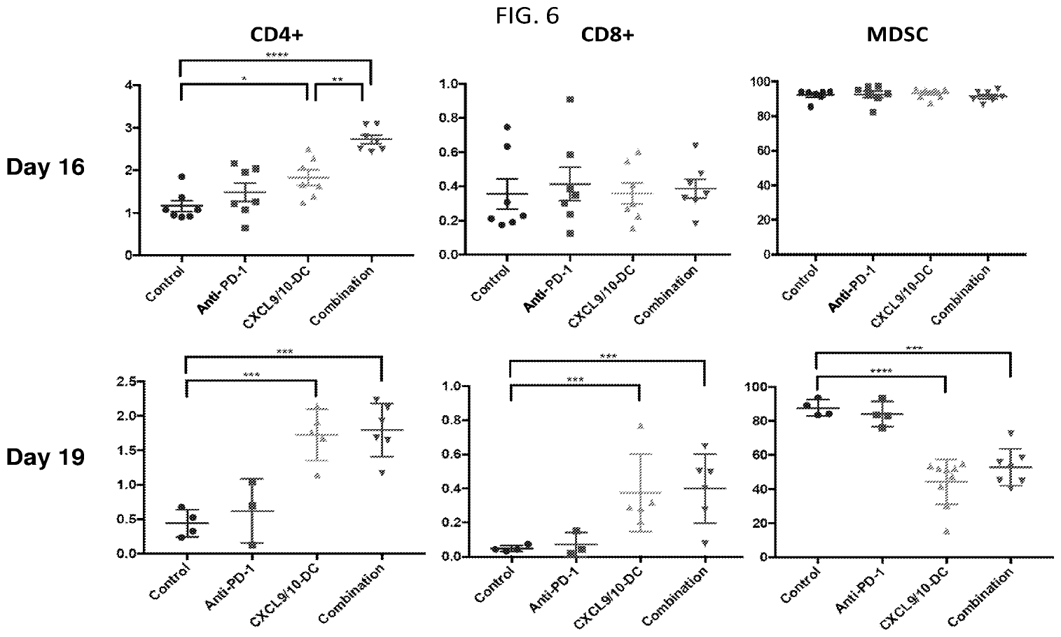

To evaluate changes in the tumor microenvironment (TME) upon treatments, the same mouse experiments as in Example 5 (FIG. 5) were performed except that tumors were harvested at days 16 and 19 post tumor inoculation, and single cell suspensions were prepared for immune phenotyping by flow cytometry (FIG. 6). At day 16, we observed increased CD4+ T cell infiltration following IT CXCL9/10-DC monotherapy (FIG. 6, top left) as well as the combination treatment, with additional increases in CD8+ T cell infiltration and a concurrent reduction of MDSC also observed at day 19 (FIG. 6, bottom left, center and right). These data support the hypothesis that IT CXCL9/10-DC can enhance T cell infiltration and function, as well as reprogram the immunosuppressive TME.

Detailed description of FIG. 6. FVB mice were SC inoculated with 1.5×10\hat{ }5 KPL-3M cells. On day 7, mice bearing <50 mm3 tumors were treated with a) vehicle; b) IP anti-PD-1 (200m/dose on days 7, 10, 13, 15); c) IT CXCL9/10-DC (10\hat{ }6 cells each/dose on days 7, 10, 13); d) combination of b) and c). On day 16 or 19 post tumor inoculation, tumors were harvested and analyzed by flow cytometry using surface markers. MDSC, myeloid-derived suppressor cell. P values were determined by non-paired t-test. *, P<0.05; **, P<0.005; ***, P<0.0005; ****, P<0.0001.

Example 7. CXCL9-DC and CXCL10-DC are Functionally Equivalent in Potentiating the Antitumor Efficacy of Anti-PD-1

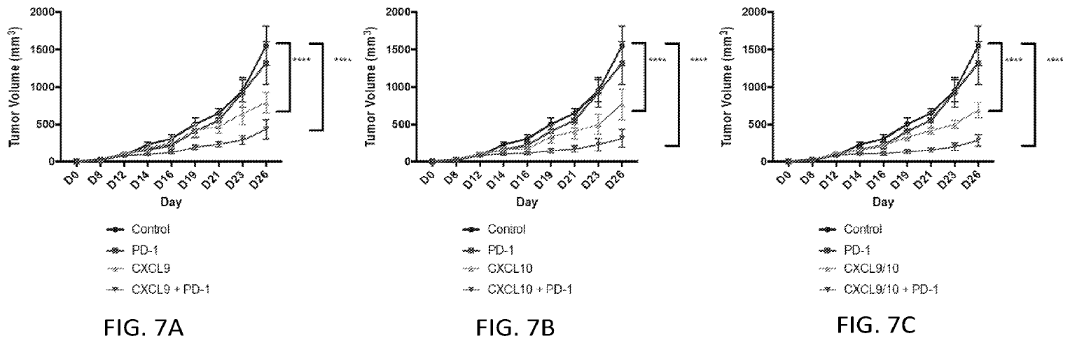

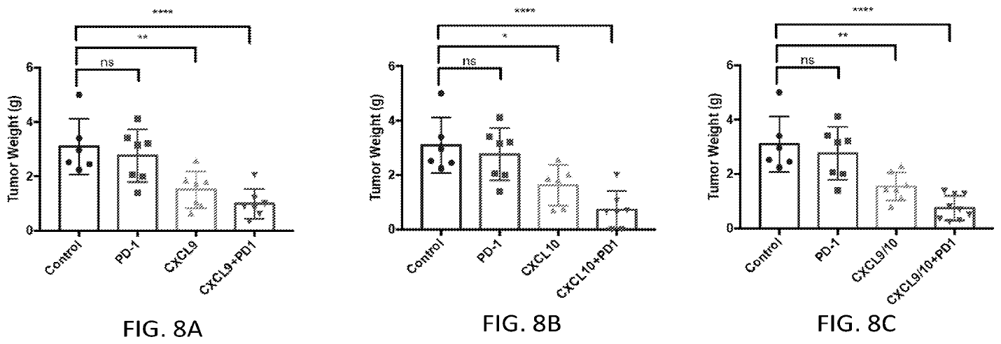

To assess whether CXCL9-DC and CXCL-10 DC have differential effect on potentiating the antitumor effect of PD-1 blockade, similar experiments as in Example 5 (FIG. 5) were performed, except that mice received IT injections of equal numbers of cells composed of CXCL9-DC alone (2×10\hat{ }6), CXCL10-DC alone (2×10\hat{ }6), or both (1:1 ratio, 10\hat{ }6 each) (FIG. 7, tumor volume; FIG. 8, tumor weight). Similar antitumor effect mediated by CXCL9-DC and CXCL10-DC, alone or in combination with anti-PD-1 were observed, suggesting that CXCL9-DC and CXCL10-DC are functionally equivalent in potentiating the antitumor efficacy of anti-PD-1.

To evaluate the synergy of the combination of CXCL9-DC, CXCL10-DC or both, and anti-PD1, doubling times of the tumor volume curves were calculated, compared to the control group doubling time, and the change and percent change in doubling time among the groups compared. A longer doubling time indicates a reduction in tumor growth. As shown in the tables below, the decrease in doubling time of the combination treatment of CXCL9-DC and anti-PD-1 was 30% greater than the sum of the decrease in doubling times of the individual treatments. The decrease in doubling time of the combination treatment of CXCL10-DC and anti-PD-1 was 2.7-fold greater than the sum of the decrease in doubling times of the individual treatments. The decrease in doubling time of the combination treatment of both CXCL9/10-DC and anti-PD-1 was 55% greater than the sum of the decrease in doubling times of the individual treatments.

| | | | | Combina- |
|---|---|---|---|---|
| | | | Sum of | tion |
| | | | anti-PD-1 | anti-PD-1 |
| Tumor volume | | | and | and |
| (doubling time) | Anti-PD-1 | CXCL9-DC | CXCL9-DC | CXCL9-DC |
| Doubling time | 0.58 | 0.810 | — | 0.896 |
| (min) | | | | |
| Control = 0.588 | | | | |
| Change in doubling | −0.01 | 0.22 | 0.21 | 0.31 |
| time from control | | | | |
| Percent change in | −1.3% | 38% | 37% | 48% |
| doubling time from | | | | |
| control | | | | |

| | | | Sum of | Combination |
|---|---|---|---|---|
| | | | anti-PD-1 | anti-PD-1 |
| Tumor volume | | | and | and |
| (doubling time) | Anti-PD-1 | CXCL10-DC | CXCL10-DC | CXCL10-DC |
| Doubling time | 0.58 | 0.720 | — | 0.905 |
| (min) | | | | |
| Control = 0.588 | | | | |
| Change in | −0.01 | 0.13 | 0.12 | 0.32 |
| doubling time | | | | |
| from control | | | | |
| Percent change | −1.3% | 22% | 21% | 54% |
| in doubling | | | | |
| time from | | | | |
| control | | | | |

| | | | Sum of | Combination |
|---|---|---|---|---|
| | | | anti-PD-1 | anti-PD-1 |
| Tumor volume | | CXCL9/10- | and | and CXCL9/ |
| (doubling time) | Anti-PD-1 | DC | CXCL9/10-DC | 10-DC |
| Doubling time | 0.58 | 0.805 | — | 0.915 |
| (min) | | | | |
| Control = 0.588 | | | | |
| Change in | −0.01 | 0.22 | 0.21 | 0.33 |
| doubling time | | | | |
| from control | | | | |
| Percent change | −1.3% | 37% | 36% | 56% |
| in doubling time | | | | |
| from control | | | | |

In a similar analysis, the change in tumor weight data are shown in the tables below. The decrease in tumor weight of the combination treatment of CXCL9-DC and anti-PD-1 was 9.3% greater than the sum of the decrease in tumor weights of the individual treatments. The decrease in tumor weight of the combination treatment of CXCL10-DC and anti-PD-1 was 31% greater than the sum of the decrease in tumor weights of the individual treatments. The decrease in tumor weight of the combination treatment of both CXCL9/10-DC and anti-PD-1 was 26% greater than the sum of the decrease in tumor weights of the individual treatments.

| | | | Sum of | Combination |
|---|---|---|---|---|
| | | | anti-PD-1 | anti-PD-1 |
| | | | and | and |
| Tumor weight | Anti-PD-1 | CXCL9-DC | CXCL9-DC | CXCL9-DC |
| Tumor weight (g) | 2.762 | 1.51 | — | 0.989 |
| Control = 3.095 | | | | |
| Change in average | −0.33 | −1.59 | −1.93 | −2.11 |
| tumor weight | | | | |
| from control | | | | |
| Percent change in | 11% | 52% | 62% | 68% |
| tumor weight | decrease | decrease | decrease | decrease |
| from control | | | | |

| | | | Sum of | Combination |
|---|---|---|---|---|
| | | | anti-PD-1 | anti-PD-1 |
| | | | and | and |
| Tumor weight | Anti-PD-1 | CXCL10-DC | CXCL10-DC | CXCL10-DC |
| Tumor weight (g) | 2.762 | 1.621 | — | 0.720 |
| Control = 3.095 | | | | |
| Change in average | −0.33 | −1.47 | −1.82 | −2.38 |
| tumor weight from | | | | |
| control | | | | |
| Percent change in | 11% | 48% | 59% | 77% |
| tumor weight from | decrease | decrease | decrease | decrease |
| control | | | | |

| | | | Sum of anti- | Combination |
|---|---|---|---|---|
| | | | PD-1 and | anti-PD-1 |
| | | CXCL9/10- | CXCL9/10- | and CXCL9/ |
| Tumor weight | Anti-PD-1 | DC | DC | 10-DC |
| Tumor weight (g) | 2.762 | 1.541 | — | 0.751 |
| Control = 3.095 | | | | |
| Change in average | −0.33 | −1.56 | −1.9 | −2.39 |
| tumor weight from | | | | |
| control | | | | |
| Percent change in | 11% | 50% | 61% | 77% |
| tumor weight from | decrease | decrease | decrease | decrease |
| control | | | | |

Detailed description of FIG. 7A, FIG. 7B and FIG. 7C. CXCL9-DC and CXCL10-DC are functionally equivalent in potentiating the antitumor efficacy of anti-PD-1, measured by tumor volume. FIG. 7A) FVB mice were SC inoculated with 1.5×10\hat{ }5 KPL-3M cells. On day 7, mice bearing <50 mm3 tumors were treated with a) vehicle; b) IP anti-PD-1 (200 μg/dose on days 7, 10, 13, 15); c) IT CXCL9-DC (2×10\hat{ }6 cells/dose on days 7, 10, 13); d) combination of b) and c). Tumor volume was recorded. FIG. 7B) Same as in FIG. 7A except that CXCL10-DC was utilized. FIG. 7C) Same as in A except that CXCL9/10-DC (10\hat{ }6 cells each/dose) was utilized. P values were determined by non-paired t-test. ****, P<0.0001.

Detailed description of FIG. 8A, FIG. 8B and FIG. 8C. CXCL9-DC and CXCL10-DC are functionally equivalent in potentiating the antitumor efficacy of anti-PD-1, measured by tumor weight. Same as in FIG. 7 except that tumor weights at the time of necropsy are presented. P values were determined by non-paired t-test. *, P<0.05; **, P<0.005; ***, P<0.0005; ****, P<0.0001. ns, not significant.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed:

1. A method of treating cancer or a solid tumor in a subject comprising
a. administering to the subject:
(i) a CXCL9 polypeptide, a CXCL10 polypeptide, or the combination thereof,
(ii) a polynucleotide encoding the CXCL9 polypeptide, a polynucleotide encoding the CXCL10 polypeptide, or the combination thereof,
(iii) a cell comprising the polynucleotide encoding the CXCL9 polypeptide, a cell comprising the polynucleotide encoding the CXCL10 polypeptide, or the combination thereof, or
(iv) any combination thereof; and
b. administering to the subject an immune checkpoint inhibitor.

2. The method of claim 1, wherein the immune checkpoint inhibitor is an antibody, optionally, a monoclonal antibody.

3. The method of claim 1 or 2, wherein the immune checkpoint inhibitor is selected from the group consisting of a CTLA-4 inhibitor, a CTLA-4 receptor inhibitor, a PD-1 inhibitor, a PD1-L1 inhibitor, a PD1-L2 inhibitor, a 4-1BB inhibitor, an OX40 inhibitor, a LAG-3 inhibitor, a TIM-3 inhibitor, or a combination thereof.

4. The method of claim 3, wherein the immune checkpoint inhibitor is a CTLA-4 inhibitor, optionally, ipilimumab or tremilimumab.

5. The method of claim 3, wherein the immune checkpoint inhibitor is a PD1 inhibitor selected from a group consisting of: Nivolumab, Pembrolizumab, Pidilizumab, Lambrolizurnab, BMS-936559, Atezolizumab, and AMP-224, AMP224, AUNP12, BGB108, MCLA134, MEDI0680, PDR001, REGN2810, SHR1210, STIA110X, STIA1110 and TSR042.

6. The method of claim 3, wherein the immune checkpoint inhibitor is a PD1-L1 inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

7. The method of claim 1, wherein the administering comprises CXCL9, CXCL10, or the combination thereof.

8. The method of claim 1 wherein the CXCL9 polypeptide comprises an amino acid sequence of SEQ ID NO: 1 or SEQ ID NO: 2; and the CXCL10 polypeptide comprises an amino acid sequence of SEQ ID NO: 3 or SEQ ID NO: 4.

9. The method of claim 1, wherein the polynucleotide encoding the CXCL9 polypeptide, CXCL10 polypeptide, or the combination thereof is inserted into a vector and the vector is administered to the subject, or the vector is introduced into an antigen presenting cell (APC) or a dendritic cell (DC) which is then administered to the subject or to the tumor site.

10. The method of claim 9, wherein the vector is an adenovirus vector, a lentiviral vector, a CMV vector, a vaccinia virus vector, a sindbis virus vector, or a herpesvirus vector.

11. The method of claim 10, wherein the adenoviral vector is a replication-deficient adenoviral vector.

12. The method of claim 1, wherein the cell comprising the polynucleotide encoding the CXCL9 polypeptide, CXCL10 polypeptide, or the combination thereof is an antigen presenting cell (APC) or a dendritic cell (DC).

13. The method of claim 12, wherein the APC is a dendritic cell.

14. The method of claim 13, wherein the dendritic cell is autologous to the subject, is from a donor or is from a cell line.

15. The method of claim 1, wherein at least or about 1×10\hat{ }6 cells comprising the polynucleotide encoding the CXCL9 polypeptide, about 1×10\hat{ }6 cells comprising the polynucleotide encoding the CXCL10 polypeptide, or the combination thereof are administered to the subject.

16. The method of claim 15, wherein the cells produce at least or about 10 ng of CXCL9 or CXCL10 per 1×10\hat{ }6 cells in a 24-hour period.

17. The method of claim 1, wherein the subject comprises a solid tumor and the cells are administered to the subject intratumorally.

18. The method of claim 17, wherein the solid tumor is a non-small cell lung carcinoma (NSCLC) solid tumor.

19. The method of claim 1, wherein the (i) CXCL9 polypeptide, the CXCL10 polypeptide, or the combination thereof, (ii) polynucleotide encoding the CXCL9 polypeptide, polynucleotide encoding the CXCL10 polypeptide, or the combination thereof, (iii) cell comprising the polynucleotide encoding the CXCL9 polypeptide, cell comprising the polynucleotide encoding the CXCL10 polypeptide, or the combination thereof, or (iv) any combination thereof, is administered to the subject prior to, about 2 weeks prior to, or at the same time as the immune checkpoint inhibitor.

20. The method of claim 1, wherein the (i) CXCL9 polypeptide, the CXCL10 polypeptide, or the combination thereof, (ii) polynucleotide encoding the CXCL9 polypeptide, polynucleotide encoding the CXCL10 polypeptide, or the combination thereof, (iii) cell comprising the polynucleotide encoding the CXCL9 polypeptide, cell comprising the polynucleotide encoding the CXCL10 polypeptide, or the combination thereof, or (iv) any combination thereof, is administered to the subject about more than once, once every two weeks, once every three weeks, or once a month.

21. The method of claim 1, wherein the immune checkpoint inhibitor is administered to the subject more than once, once every 2 weeks, once every 3 weeks, or once a month.

22. The method of claim 1, wherein the (i) CXCL9 polypeptide, the CXCL10 polypeptide, or the combination thereof, (ii) polynucleotide encoding the CXCL9 polypeptide, polynucleotide encoding the CXCL10 polypeptide, or the combination thereof, (iii) cell comprising the polynucleotide encoding the CXCL9 polypeptide, cell comprising the polynucleotide encoding the CXCL10 polypeptide, or the combination thereof, or (iv) any combination thereof, and the immune checkpoint inhibitor, are independently administered by a route selected from intratumorally, intravenously, intra-arterially, intraperitoneally, intranasally, intramuscularly, intradermally or subcutaneously, or via CT-guided or bronchoscopic IT injection.

23. The method of claim 1, wherein the (i) CXCL9 polypeptide, the CXCL10 polypeptide, or the combination thereof, (ii) polynucleotide encoding the CXCL9 polypeptide, polynucleotide encoding the CXCL10 polypeptide, or the combination thereof, (iii) cell comprising the polynucleotide encoding the CXCL9 polypeptide, cell comprising the polynucleotide encoding the CXCL10 polypeptide, or the combination thereof, or (iv) any combination thereof, is administered intratumorally and the immune checkpoint inhibitor is administered intravenously.

24. The method of any one of claims 1-23, wherein the subject has a high mutational burden tumor.

25. The method of claim 24, wherein the high mutational burden is determined by a biopsy of the tumor.

26. The method of claim 24, wherein tumor-associated neoantigens are determined.

27. The method of claim 24, wherein the efficacy of combination therapy is followed by elucidation of the neoantigen landscape of the tumor.

28. The method of claims 24, wherein the tumor comprises a mutation selected from KRAS, TP53 (KP) or STK11/LKB1, or any combination thereof.

29. The method of claim 24, wherein the tumor has intratumoral heterogeneity.

30. The method of claim 24, wherein the tumor mutational burden is determined by diagnostic assay selected from FoundationOne CDx™, FoundationOne®, FoundationAct®, and FoundationOne®Heme.

31. The method of claim 24, wherein the tumor does not have an activating mutation in the epidermal growth factor receptor or an anaplastic lymphoma kinase gene (ALK) fusion.

32. The method of claim 24, wherein the somatic mutational load and tumor-associated neoantigens before, during and after treatment are used to initiate, prescribe and monitor therapy.

33. The method of any one of claims 1-23, wherein the subject has a low mutational burden tumor.

34. The method of claim 33, wherein the low mutational burden is determined by a biopsy of the tumor.

35. The method of claim 33, wherein tumor-associated neoantigens are determined.

36. The method of claim 33, wherein the efficacy of combination therapy is followed by elucidation of the neoantigen landscape of the tumor.

37. The method of claims 33, wherein the tumor comprises a mutation selected from KRAS, TP53 (KP) or STK11/LKB1, or any combination thereof.

38. The method of claim 33, wherein the tumor has intratumoral heterogeneity.

39. The method of claim 33, wherein the tumor mutational burden is determined by diagnostic assay selected from FoundationOne CDx™, FoundationOne®, FoundationAct®, and FoundationOne®Heme.

40. The method of claim 33, wherein the tumor does not have an activating mutation in the epidermal growth factor receptor or an anaplastic lymphoma kinase gene (ALK) fusion.

41. The method of claim 33, wherein the somatic mutational load and tumor-associated neoantigens before, during and after treatment are used to initiate, prescribe and monitor therapy.

42. A method for treating cancer or reducing the reoccurrence of a high mutational burden cancer in a subject in need thereof comprising administering an effective amount of a combination therapy comprising a) dendritic cells comprising an CXCL9 vector and dendritic cells comprising a CXCL10 vector, and b) an effective amount of anti-PD-1 antibody.

43. The method of claim 42, wherein the dendritic cells comprising an CXCL9 vector and dendritic cells comprising a CXCL10 vector are administered on days 0, 21, and 42, and an effective amount of anti-PD-1 antibody is administered every three weeks starting on day 0, optionally wherein the vector is an lentiviral vector.

44. The method of claim 42, wherein the high mutational burden is determined by a biopsy of the tumor.

45. The method of claim 42, wherein tumor-associated neoantigens are determined.

46. The method of claim 42, wherein the efficacy of combination therapy is followed by elucidation of the neoantigen landscape of the tumor.

47. The method of claims 42, wherein the tumor comprises a mutation selected from KRAS, TP53 (KP) or STK11/LKB1, or any combination thereof.

48. The method of claim 42, wherein the tumor has intratumoral heterogeneity.

49. The method of claim 42, wherein the tumor mutational burden is determined by diagnostic assay selected from FoundationOne CDx™, FoundationOne®, FoundationAct®, and FoundationOne®Heme.

50. The method of claim 42, wherein the tumor does not have an activating mutation in the epidermal growth factor receptor or an anaplastic lymphoma kinase gene (ALK) fusion.

51. The method of claim 42, wherein the somatic mutational load and tumor-associated neoantigens before, during and after treatment are used to initiate, prescribe and monitor therapy.

52. A method for treating cancer or reducing the reoccurrence of a low mutational burden cancer in a subject in need thereof comprising administering an effective amount of a combination therapy comprising a) dendritic cells comprising an CXCL9 vector and dendritic cells comprising a CXCL10 vector, and b) an effective amount of anti-PD-1 antibody.

53. The method of claim 52, wherein dendritic cells comprising an CXCL9 vector and dendritic cells comprising a CXCL10 vector are administered on days 0, 21, and 42, and an effective amount of anti-PD-1 antibody is administered every three weeks starting on day 0, optionally wherein the vector is an lentiviral vector.

54. The method of claim 52, wherein the low mutational burden is determined by a biopsy of the tumor.

55. The method of claim 52, wherein tumor-associated neoantigens are determined.

56. The method of claim 52, wherein the efficacy of combination therapy is followed by elucidation of the neoantigen landscape of the tumor.

57. The method of claims 52, wherein the tumor comprises a mutation selected from KRAS, TP53 (KP) or STK11/LKB1, or any combination thereof.

58. The method of claim 52, wherein the tumor has intratumoral heterogeneity.

59. The method of claim 52, wherein the tumor mutational burden is determined by diagnostic assay selected from FoundationOne CDx™, FoundationOne®, FoundationAct®, and FoundationOne®Heme.

60. The method of claim 52, wherein the tumor does not have an activating mutation in the epidermal growth factor receptor or an anaplastic lymphoma kinase gene (ALK) fusion.

61. The method of claim 52 wherein the somatic mutational load and tumor-associated neoantigens before, during and after treatment are used to initiate, prescribe and monitor therapy.

62. An antigen presenting cell comprising a vector a comprises a polynucleotide encoding CXCL9, CXCL10, or the combination thereof.

63. The antigen presenting cell of claim 62, wherein the antigen presenting cell is a dendritic cell.

64. The antigen presenting cell of claim 62, wherein the CXCL9 polypeptide comprises an amino acid sequence of SEQ ID NO: 1 or SEQ ID NO: 2.

65. The antigen presenting cell of claim 62, wherein the CXCL9 polynucleotide sequence comprises SEQ ID NO: 5 or SEQ ID NO: 6.

66. The antigen presenting cell of claim 62, wherein the CXCL10 polypeptide comprises an amino acid sequence of SEQ ID NO: 3 or SEQ ID NO: 4.

67. The antigen presenting cell of claim 62, wherein the CXCL10 polynucleotide sequence comprises SEQ ID NO: 7 or SEQ ID NO: 8.
